(19) United States
(12) Patent Application Publication
(10) Pub. No.: US 2026/0124435 A1
(43) Pub. Date: May 7, 2026

(54) BALLOON FOR BALLOON CATHETER AND BALLOON CATHETER

(71) Applicant: KANEKA CORPORATION, Osaka (JP)

(72) Inventors: Masahiro Kojima, Osaka (JP); Takahisa Hamabuchi, Osaka (JP); Masato Tsueda, Osaka (JP)

(73) Assignee: KANEKA CORPORATION, Osaka (JP)

(21) Appl. No.: 19/425,135

(22) Filed: Dec. 18, 2025

Related U.S. Application Data
(63) Continuation of application No. PCT/JP2024/023144, filed on Jun. 26, 2024.

(30) Foreign Application Priority Data
Jul. 3, 2023 (JP) 2023-109026
Jul. 3, 2023 (JP) 2023-109027

(51) Int. Cl. A61M 25/10; A61B 17/22; A61B 17/3207

(52) U.S. Cl. CPC A61M 25/1002; A61B 17/22; A61B 17/320725; A61M 25/104; A61B 2017/22001; A61B 2017/22065; A61M 2025/1086

(57) ABSTRACT

A balloon for a balloon catheter including a straight tube portion provided with a ridge extending in a longitudinal axis direction is provided. A specific notch is formed in the ridge. The ridge is divided into a plurality of ridge segments by the specific notch. In a distal surface of the ridge segment adjacent to a proximal side of the specific notch, a normal vector A on a line of intersection with a virtual plane formed by the longitudinal axis direction and a radial direction and passing through a top portion of the ridge is oriented toward a distal side on one side of the virtual plane. In a proximal surface of the ridge segment adjacent to the distal side of the specific notch, a normal vector B on the line of intersection with the virtual plane is oriented toward the proximal side on another side of the virtual plane.

### TECHNICAL FIELD

One or more embodiments of the present invention relate to a balloon for a balloon catheter and a balloon catheter including the balloon.

### BACKGROUND

It is known that various diseases occur when a blood vessel, which is a flow path through which blood circulates in the body, becomes narrowed and the blood circulation stagnates. In particular, when stenosis occurs in a coronary artery that supplies blood to the heart, it may cause serious diseases such as angina pectoris and myocardial infarction. As one of the methods for treating such a stenosed site of a blood vessel, there is angioplasty (PTA, PTCA, etc.) that expands a stenosed site by using a balloon catheter.

Balloon catheters are known in each of which ridges are provided on a surface of a balloon (for example, PTLs 1 to 3). If such a balloon catheter is used, when a balloon is inflated, ridges of the balloon can bite into a stenosed site, and the stenosed site can be effectively expanded. On the other hand, a balloon provided with ridges tends to have high rigidity at its portions where the ridges are provided, and its flexibility in the longitudinal axis direction thereof is likely to decrease. In contrast, a balloon catheter in which notches are formed in ridges on a balloon surface is known (for example, PTLs 4 and 5). If such a balloon catheter is used, it is possible to maintain the flexibility of a balloon in the longitudinal axis direction of the balloon even if the balloon is provided with ridges.

### Patent Literature

PTL 1: International Publication No. 2020/250611

PTL 2: Japanese Unexamined Patent Application Publication No. 2009-112361

PTL 3: Japanese Unexamined Patent Application Publication No. 2013-176507

PTL 4: International Publication No. 2012/099950

PTL 5: International Publication No. 2020/255923

A balloon with ridges provided on its surface and notches formed in the ridges can exhibit a scoring function by the ridges, while ensuring the flexibility of the balloon in the longitudinal axis direction of the balloon by the notches formed in the ridges. Since blood vessels have significantly curved portions, it is desirable that a balloon provided with ridges can be smoothly inserted even through such curved portions. Therefore, it is more desirable to further increase the flexibility of the balloon.

### SUMMARY

One or more embodiments of the present invention have been made in view of the above-mentioned circumstances, and a balloon for a balloon catheter that is a balloon provided with at least one ridge on its surface and that can achieve increased flexibility in a longitudinal axis direction, and a balloon catheter including the balloon are provided.

A balloon for a balloon catheter and a balloon catheter including the balloon, both according to one or more embodiments of the present invention and having addressed the above, are as follows.

[1] A balloon for a balloon catheter having a longitudinal axis direction extending from a proximal side to a distal side and a radial direction orthogonal to the longitudinal axis direction.

The balloon includes a straight tube portion, a proximal tapered portion located on a proximal side of the straight tube portion, and a distal tapered portion located on a distal side of the straight tube portion.

The straight tube portion includes a balloon main body having a cylindrical shape, and a ridge provided on an outer surface of the balloon main body, the ridge protruding outward in a radial direction and extending in the longitudinal axis direction.

At least one notch is formed in the ridge, and the ridge is divided into a plurality of ridge segments by the at least one notch.

The at least one notch includes at least one specific notch that satisfies Requirement A below.

### (Requirement A)

A distal surface of one of the ridge segments (hereinafter referred to as a “proximal ridge segment”) that is adjacent to a proximal side of the at least one specific notch has a normal vector A on a line of intersection with a virtual plane formed by the longitudinal axis direction and the radial direction and passing through a top portion of the ridge, the normal vector A being oriented toward a distal side on one side of the virtual plane.

A proximal surface of another one of the ridge segments (hereinafter referred to as a “distal ridge segment”) that is adjacent to a distal side of the at least one specific notch has a normal vector B on a line of intersection with the virtual plane, the normal vector B being oriented toward a proximal side on another side of the virtual plane.

[2] A balloon for a balloon catheter having a longitudinal axis direction extending from a proximal side to a distal side and a radial direction orthogonal to the longitudinal axis direction.

The balloon comprises a straight tube portion, a proximal tapered portion located on a proximal side of the straight tube portion, and a distal tapered portion located on a distal side of the straight tube portion.

The straight tube portion includes a balloon main body having a cylindrical shape, and a ridge provided on an outer surface of the balloon main body, the ridge protruding outward in a radial direction and extending in the longitudinal axis direction.

A notch is formed in the ridge, and the ridge is divided into a plurality of ridge segments by the notch.

The notch includes a specific notch that satisfies Requirement B below.

### (Requirement B)

In a plan view as viewed from a top portion of the ridge, a distal end edge of one of the ridge segments (hereinafter referred to as a “proximal ridge segment”) that is adjacent to a proximal side of the specific notch has a protruding shape protruding toward a distal side, and a proximal end edge of another one of the ridge segments (hereinafter referred to as a “distal ridge segment”) that is adjacent to a distal side of the specific notch has a protruding shape protruding toward a proximal side.

[3] The balloon according to [1], wherein the ridge has a plurality of the specific notches arranged in the longitudinal axis direction, and wherein, when one side of the virtual plane in the ridge is defined as a first side and another side of the virtual plane is defined as a second side, in all of the specific notches provided in the ridge, the normal vector A is oriented toward a distal side on the first side, and the normal vector B is oriented toward a proximal side on the second side.
[4] The balloon according to [1], wherein the ridge has a plurality of the specific notches arranged in the longitudinal axis direction, and wherein, when one side of the virtual plane in the ridge is defined as a first side and another side of the virtual plane is defined as a second side, the specific notches having the normal vector A oriented toward a distal side on the first side and the normal vector B oriented toward a proximal side on the second side and the specific notches having the normal vector A oriented toward a distal side on the second side and the normal vector B oriented toward a proximal side on the first side are provided in the ridge in such a manner as to be alternately arranged in a longitudinal axis direction.
[5] The balloon according to [1], [3], or [4], wherein a distal surface of the proximal ridge segment and a proximal surface of the distal ridge segment are closest to each other in the longitudinal axis direction at least on a line of intersection with the virtual plane.
[6] The balloon according to [5], wherein a separation distance in the longitudinal axis direction between a distal surface of the proximal ridge segment and a proximal surface of the distal ridge segment is formed in such a manner as to remain unchanged or to increase with increasing distance from the virtual plane.
[7] The balloon according to [6], wherein a separation distance in the longitudinal axis direction between a distal surface of the proximal ridge segment and a proximal surface of the distal ridge segment is formed in such a manner as to increase at least in part with increasing distance from the virtual plane.
[8] The balloon according to any one of [1] and [3] to [7], wherein a distal surface of the proximal ridge segment is inclined proximally outward in a radial direction on a line of intersection with the virtual plane, and wherein a proximal surface of the distal ridge segment is inclined distally outward in a radial direction on a line of intersection with the virtual plane.
[9] The balloon according to any one of [1] and [3] to [8], wherein an angle formed between a projection vector AP of the normal vector A onto an outer surface of the balloon main body and the longitudinal axis direction toward a distal side is 20° or more and 70° or less, and wherein an angle formed between a projection vector BP of the normal vector B onto an outer surface of the balloon main body and the longitudinal axis direction toward a proximal side is 20° or more and 70° or less.
[10] The balloon according to [2], wherein, in a plan view as viewed from a top portion of the ridge, a most distal portion of the distal end edge of the proximal ridge segment is on a virtual straight line passing through a top portion of the ridge and extending in the longitudinal axis direction, and/or wherein, in a plan view as viewed from a top portion of the ridge, a most proximal portion of the proximal end edge of the distal ridge segment is on a virtual straight line passing through a top portion of the ridge and extending in the longitudinal axis direction.
[11] The balloon according to [2] or [10], wherein the protruding shape of the distal end edge of the proximal ridge segment includes a curved portion curved toward a distal side, and/or wherein the protruding shape of the proximal end edge of the distal ridge segment includes a curved portion curved toward a proximal side.
[12] The balloon according to [2], [10], or [11] wherein, in a plan view as viewed from a top portion of the ridge, the protruding shape of the distal end edge of the proximal ridge segment includes a proximal first straight portion located on one side of a virtual straight line passing through a top portion of the ridge and extending in the longitudinal axis direction, and a proximal second straight portion located on another side of the virtual straight line, and the proximal first straight portion and the proximal second straight portion extend toward a proximal side away from the virtual straight line with increasing distance, and/or wherein, in a plan view as viewed from a top portion of the ridge, the protruding shape of the proximal end edge of the distal ridge segment includes a distal first straight portion located on one side of the virtual straight line and a distal second straight portion located on another side of the virtual straight line, and the distal first straight portion and the distal second straight portion extend toward a distal side away from the virtual straight line with increasing distance.
[13] The balloon according to [12], wherein, in a plan view as viewed from a top portion of the ridge, the protruding shape of the distal end edge of the proximal ridge segment further includes a proximal third straight portion between the proximal first straight portion and the proximal second straight portion, and the proximal first straight portion and the proximal second straight portion are located on a proximal side with respect to the proximal third straight portion, and/or wherein, in a plan view as viewed from a top portion of the ridge, the protruding shape of the proximal end edge of the distal ridge segment further includes a distal third straight portion between the distal first straight portion and the distal second straight portion, and the distal first straight portion and the distal second straight portion are located on a distal side with respect to the distal third straight portion.
[14] The balloon according to [13], wherein the proximal third straight portion and/or the distal third straight portion extends perpendicularly to the longitudinal axis direction.
[15] The balloon according to [12], wherein a distal end of the proximal first straight portion is connected to a distal end of the proximal second straight portion, and/or wherein a proximal end of the distal first straight portion is connected to a proximal end of the distal second straight portion.
[16] The balloon according to any one of [2] and [10] to [15], wherein, in a cross section taken along a longitudinal axis direction and passing through a top portion of the ridge, a distal outer edge of the proximal ridge segment extends in such a manner as to be inclined proximally outward in a radial direction, and/or wherein, in a cross section taken along a longitudinal axis direction and passing through a top portion of the ridge, a proximal outer edge of the distal ridge segment extends in such a manner as to be inclined distally outward in a radial direction.
[17] The balloon according to any one of [1] to [16], wherein the ridge is made of a resin, a metal, or a combination thereof.
[18] A balloon catheter comprising the balloon according to any one of [1] to [17].

In the balloon for a balloon catheter of one or more embodiments of the present invention, the ridge is provided on the outer surface of the straight tube portion of the balloon, and thus, when a balloon catheter including the balloon is used and the balloon is inflated at a stenosed site or the like of a blood vessel, the ridge can bite into the stenosed site or the like, and the stenosed site can be effectively expanded. In addition, at least one notch is formed in the ridge of the balloon, and at least one of the at least one notch is the specific notch described above. Thus, the flexibility of the balloon can be enhanced.

### BRIEF DESCRIPTION OF DRAWINGS

FIG. 1 illustrates a configuration example of a balloon catheter according to one or more embodiments of the present invention and illustrates a side view of the balloon catheter.

FIG. 2 is a cross-sectional view of the balloon catheter taken along line II-II of FIG. 1.

FIG. 3 is a cross-sectional view of the balloon catheter taken along line III-III of FIG. 1.

FIG. 4 illustrates an example of a perspective view of a balloon according to a first embodiment included in the balloon catheter.

FIG. 5 illustrates an example of a perspective view of a balloon according to a second embodiment included in a balloon catheter.

FIG. 6 is a vertical cross-sectional view of a straight tube portion of the balloon illustrated in FIG. 4 and FIG. 5 taken perpendicular to a longitudinal axis direction.

FIG. 7 is a cross-sectional view of a ridge of the balloon illustrated in FIG. 6 taken perpendicular to the longitudinal axis direction.

FIG. 8(a) and FIG. 8(b) illustrate a configuration example of a specific notch formed in the ridge of the balloon according to the first embodiment and a ridge segment adjacent thereto, FIG. 8(a) being a perspective view of an end portion of the ridge segment, FIG. 8(b) being a plan view of the end portion of the ridge segment as viewed from the side on which a top portion of the ridge is present.

FIG. 9(a) and FIG. 9(b) illustrate a configuration example of the specific notch formed in the ridge of the balloon according to the first embodiment and the ridge segment adjacent thereto, FIG. 9(a) being a perspective view of the end portion of the ridge segment, FIG. 9(b) being a plan view of the end portion of the ridge segment as viewed from the side on which the top portion of the ridge is present.

FIG. 10(a) and FIG. 10(b) illustrate a configuration example of the specific notch formed in the ridge of the balloon according to the first embodiment and the ridge segment adjacent thereto, FIG. 10(a) being a perspective view of the end portion of the ridge segment, FIG. 10(b) being a plan view of the end portion of the ridge segment as viewed from the side on which the top portion of the ridge is present.

FIG. 11(a) and FIG. 11(b) illustrate a configuration example of the specific notch formed in the ridge of the balloon according to the first embodiment and the ridge segment adjacent thereto, FIG. 11(a) being a perspective view of the end portion of the ridge segment, FIG. 11(b) being a plan view of the end portion of the ridge segment as viewed from the side on which the top portion of the ridge is present.

FIG. 12(a) and FIG. 12(b) illustrate a configuration example of the specific notch formed in the ridge of the balloon according to the first embodiment and the ridge segment adjacent thereto, FIG. 12(a) being a perspective view of the end portion of the ridge segment, FIG. 12(b) being a plan view of the end portion of the ridge segment as viewed from the side on which the top portion of the ridge is present.

FIG. 13(a) and FIG. 13(b) illustrate a configuration example of the specific notch formed in the ridge of the balloon according to the first embodiment and the ridge segment adjacent thereto, FIG. 13(a) being a perspective view of the end portion of the ridge segment, FIG. 13(b) being a plan view of the end portion of the ridge segment as viewed from the side on which the top portion of the ridge is present.

FIG. 14(a) and FIG. 14(b) illustrate a configuration example of the specific notch formed in the ridge of the balloon according to the first embodiment and the ridge segment adjacent thereto, FIG. 14(a) being a perspective view of the end portion of the ridge segment, FIG. 14(b) being a plan view of the end portion of the ridge segment as viewed from the side on which the top portion of the ridge is present.

FIG. 15(a) and FIG. 15(b) illustrate a configuration example of the specific notch formed in the ridge of the balloon according to the first embodiment and the ridge segment adjacent thereto, FIG. 15(a) being a perspective view of the end portion of the ridge segment, FIG. 15(b) being a plan view of the end portion of the ridge segment as viewed from the side on which the top portion of the ridge is present.

FIG. 16 is a plan view of the balloon according to the first embodiment as viewed from the side on which the top portion of the ridge is present, and illustrates a configuration example of the ridge in which a plurality of specific notches are provided in the longitudinal axis direction.

FIG. 17 is a plan view of the balloon according to the first embodiment as viewed from the side on which the top portion of the ridge is present, and illustrates another configuration example of the ridge in which a plurality of specific notches are provided in the longitudinal axis direction.

FIG. 18(a) and FIG. 18(b) illustrate a configuration example of a specific notch formed in a ridge of a balloon according to a second embodiment and a ridge segment adjacent thereto, FIG. 18(a) being a perspective view of an end portion of the ridge segment, FIG. 18(b) being a plan view of the end portion of the ridge segment as viewed from the side on which a top portion of the ridge is present.

FIG. 19(a) and FIG. 19(b) illustrate a configuration example of the specific notch formed in the ridge of the balloon according to the second embodiment and the ridge segment adjacent thereto, FIG. 19(a) being a perspective view of the end portion of the ridge segment, FIG. 19(b) being a plan view of the end portion of the ridge segment as viewed from the side on which the top portion of the ridge is present.

FIG. 20(a) and FIG. 20(b) illustrate a configuration example of the specific notch formed in the ridge of the balloon according to the second embodiment and the ridge segment adjacent thereto, FIG. 20(a) being a perspective view of the end portion of the ridge segment, FIG. 20(b) being a plan view of the end portion of the ridge segment as viewed from the side on which the top portion of the ridge is present.

FIG. 21(a) and FIG. 21(b) illustrate a configuration example of the specific notch formed in the ridge of the balloon according to the second embodiment and the ridge segment adjacent thereto, FIG. 21(a) being a perspective view of the end portion of the ridge segment, FIG. 21(b) being a plan view of the end portion of the ridge segment as viewed from the side on which the top portion of the ridge is present.

FIG. 22(a) and FIG. 22(b) illustrate a configuration example of the specific notch formed in the ridge of the balloon according to the second embodiment and the ridge segment adjacent thereto, FIG. 22(a) being a perspective view of the end portion of the ridge segment, FIG. 22(b) being a plan view of the end portion of the ridge segment as viewed from the side on which the top portion of the ridge is present.

FIG. 23(a) and FIG. 23(b) illustrate a configuration example of the specific notch formed in the ridge of the balloon according to the second embodiment and the ridge segment adjacent thereto, FIG. 23(a) being a perspective view of the end portion of the ridge segment, FIG. 23(b) being a plan view of the end portion of the ridge segment as viewed from the side on which the top portion of the ridge is present.

FIG. 24(a) and FIG. 24(b) illustrate a configuration example of the specific notch formed in the ridge of the balloon according to the second embodiment and the ridge segment adjacent thereto, FIG. 24(a) being a perspective view of the end portion of the ridge segment, FIG. 24(b) being a plan view of the end portion of the ridge segment as viewed from the side on which the top portion of the ridge is present.

FIG. 25(a) and FIG. 25(b) illustrate a configuration example of the specific notch formed in the ridge of the balloon according to the second embodiment and the ridge segment adjacent thereto, FIG. 25(a) being a perspective view of the end portion of the ridge segment, FIG. 25(b) being a plan view of the end portion of the ridge segment as viewed from the side on which the top portion of the ridge is present.

FIG. 26 illustrates an example of a cross-sectional view, taken along the longitudinal axis direction, of the ridge of the balloon and a specific notch.

FIG. 27 illustrates another example of a cross-sectional view, taken along the longitudinal axis direction, of the ridge of the balloon and the specific notch.

FIG. 28 illustrates another example of a cross-sectional view, taken along the longitudinal axis direction, of the ridge of the balloon and the specific notch.

FIG. 29 illustrates another example of a cross-sectional view, taken along the longitudinal axis direction, of the ridge of the balloon and the specific notch.

FIG. 30 illustrates another example of a perspective view of the balloon according to the first embodiment included in the balloon catheter.

FIG. 31 illustrates another example of a perspective view of the balloon according to the first embodiment included in the balloon catheter.

FIG. 32 illustrates another example of a perspective view of the balloon according to the second embodiment included in the balloon catheter.

FIG. 33 illustrates another example of a perspective view of the balloon according to the second embodiment included in the balloon catheter.

FIG. 34 illustrates another example of a cross-sectional view of the ridge of the balloon taken perpendicular to the longitudinal axis direction.

### DETAILED DESCRIPTION

Hereinafter, one or more embodiments of the present invention will be described in detail based on embodiments below. However, needless to say, the present invention is not limited to the embodiment below and may be appropriately modified within the scope of the present invention described above and below, and all of the modifications are included in the technical scope of the present invention. In each drawing, hatching, the reference signs of components, and the like may sometimes be omitted for convenience. In such cases, reference may be made to the specification or other drawings. Additionally, the dimensions of various components in the drawings may differ from the actual dimensions as priority is given to facilitating understanding of the features of one or more embodiments of the present invention.

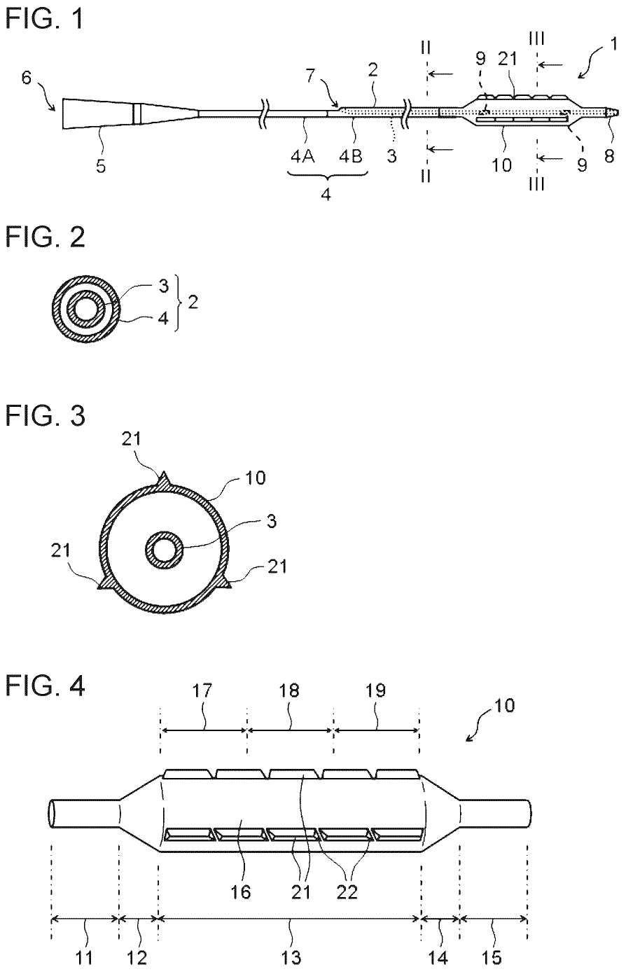

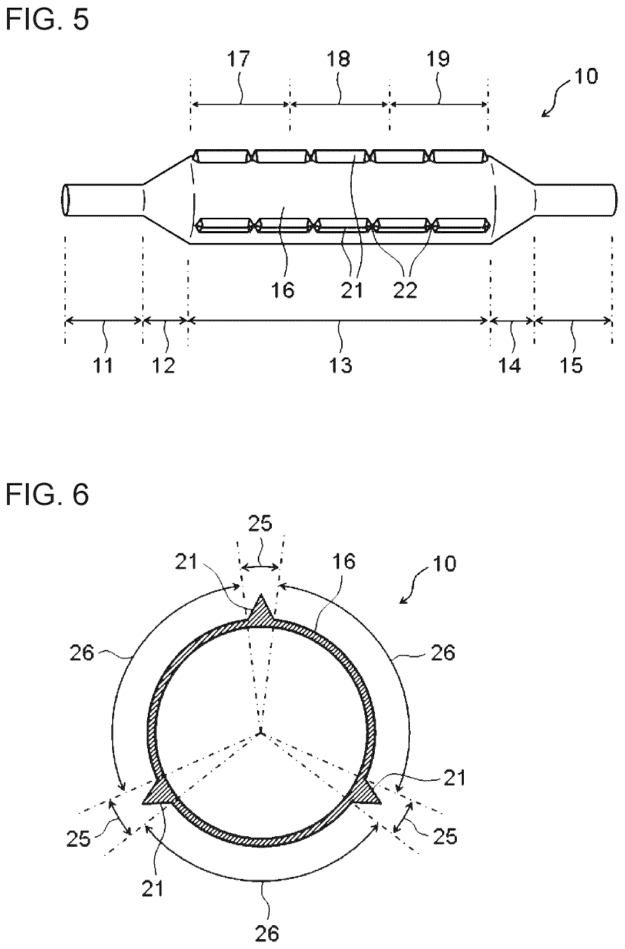

A configuration example of a balloon for a balloon catheter according to one or more embodiments of the present invention and a configuration example of a balloon catheter according to one or more embodiments of the present invention that includes the balloon will be described with reference to the drawings. FIGS. 1 to 5 illustrate configuration examples of a balloon catheter. FIG. 1 is a side view of the balloon catheter. FIG. 2 is a cross-sectional view of the balloon catheter taken along line II-II of FIG. 1. FIG. 3 is a cross-sectional view of the balloon catheter taken along line III-III of FIG. 1. FIGS. 4 and 5 each illustrate an example of a perspective view of a balloon included in the balloon catheter. FIG. 1 illustrates an example of the configuration of a rapid-exchange balloon catheter. FIG. 4 is a perspective view of the balloon according to the first embodiment. A specific notch that satisfies Requirement A, which will be described later, is formed in a ridge provided on a surface of the balloon. FIG. 5 is a perspective view of the balloon according to the second embodiment. A specific notch that satisfies Requirement B, which will be described later, is formed in a ridge provided on a surface of the balloon.

A balloon catheter 1 includes a shaft 2 and a balloon 10 provided outside the shaft 2. The balloon catheter 1 has a proximal side and a distal side, and the balloon 10 is provided on a distal portion of the shaft 2. The proximal side of the balloon catheter 1 refers to a direction toward a user's (operator's) hand with respect to a direction in which the balloon catheter 1 extends, and the distal side of the balloon catheter 1 refers to a direction opposite to the proximal side, that is, the direction toward a treatment target. The direction from the proximal side to the distal side of the balloon catheter 1 will be referred to as a longitudinal axis direction.

The balloon catheter 1 is configured such that a fluid is supplied to the inside of the balloon 10 through the shaft 2, and inflation and deflation of the balloon 10 can be controlled by using an indeflator (a pressure regulator for a balloon). The fluid may be a pressurized fluid that is pressurized by a pump or the like. Hereinafter, the fluid to be supplied to the inside of the balloon 10 will be referred to as a “balloon inflation fluid”.

The shaft 2 includes, for example, an inner shaft 3 and an outer shaft 4. The inner shaft 3 is disposed in the lumen of the outer shaft 4. The inner shaft 3 can function as an insertion path for a guidewire along which the shaft 2 is advanced, and when the balloon catheter 1 is used, the guidewire is inserted into the lumen of the inner shaft 3. The space between the inner shaft 3 and the outer shaft 4 can function as a flow path of the balloon inflation fluid.

In the rapid-exchange balloon catheter 1, a guidewire port 7 is provided at an intermediate position between the distal side and the proximal side of the shaft 2. The proximal end of the inner shaft 3 is connected to the guidewire port 7, and the distal end of the inner shaft 3 extends to the distal portion of the shaft 2, so that the insertion path for the guidewire is formed in such a manner as to extend from the guidewire port 7 to the distal portion of the shaft 2.

The outer shaft 4 may include a proximal outer shaft 4A and a distal outer shaft 4B, and in this case, the inner shaft 3 may be disposed in the lumen of the distal outer shaft 4B. The proximal outer shaft 4A and the distal outer shaft 4B may be made of the same material or may be made of different materials. For example, it is preferable that the proximal outer shaft 4A be made of a resin or a metal, and that the distal outer shaft 4B be made of a resin. The outer shaft 4 is not necessarily divided into the proximal outer shaft 4A and the distal outer shaft 4B and may be formed of a single member. Alternatively, the proximal outer shaft 4A and the distal outer shaft 4B may each be further formed of a plurality of tube members.

A hub 5 may be provided on the proximal side of the shaft 2. The hub 5 may include a fluid injection portion 6 in communication with the flow path of the balloon inflation fluid in the shaft 2. The balloon 10, the shaft 2 (the inner shaft 3 and the outer shaft 4), and the hub 5 can be joined to each other by using joining means that is known in the related art, such as an adhesive or thermal welding.

Although not illustrated in the drawings, the balloon catheter may be an over-the-wire balloon catheter in which an inner shaft extends from a distal portion of a shaft to a proximal portion of the shaft and in which an insertion path for a guidewire is formed in such a manner as to extend from the distal side of the shaft to the proximal side of the shaft. In this case, it is preferable that the flow path of the balloon inflation fluid and the insertion path for the guidewire, which are provided in the shaft, extend to the hub, and that the hub be configured to include the fluid injection portion communicating with the flow path of the balloon inflation fluid and a treatment portion communicating with the insertion path for the guidewire. It is preferable that the hub have a bifurcated structure in which the fluid injection portion is provided in one of bifurcated portions and in which the treatment portion is provided in the other bifurcated portion.

The outer surface of the shaft 2 may be coated. In the rapid-exchange balloon catheter 1, one or both of the outer surface of the proximal outer shaft 4A and the outer surface of the distal outer shaft 4B may be coated, or the outer surfaces of both the proximal outer shaft 4A and the distal outer shaft 4B may be coated. In the over-the-wire balloon catheter, the outer surface of the outer shaft may be coated as appropriate.

The coating can be a hydrophilic coating or a hydrophobic coating, depending on the purpose. The outer surface of the shaft 2 can be coated by immersing the shaft 2 in a hydrophilic coating agent or a hydrophobic coating agent, by applying a hydrophilic coating agent or a hydrophobic coating agent to the outer surface of the shaft 2, or by coating the outer surface of the shaft 2 with a hydrophilic coating agent or a hydrophobic coating agent. The coating agent may contain a drug or an additive.

Examples of the hydrophilic coating agent include hydrophilic polymers such as polyvinyl alcohol, polyethylene glycol, polyacrylamide, polyvinylpyrrolidone, and methyl vinyl ether-maleic anhydride copolymer, and hydrophilic coating agents and the like made of any combination of these.

Examples of the hydrophobic coating agent include polytetrafluoroethylene (PTFE), fluorinated ethylene propylene (FEP), perfluoroalkoxyalkane (PFA), silicone oil, hydrophobic urethane resin, carbon coating, diamond coating, diamond-like carbon (DLC) coating, ceramic coating, and substances and the like with low surface free energy terminated with alkyl groups or perfluoroalkyl groups.

A distal tip 8 may be provided at a distal end portion of the balloon catheter 1. The distal tip 8 may be provided, as a separate component from the inner shaft 3, at a position further toward the distal side than the distal end of the inner shaft 3, or the inner shaft 3 may extend to a position further toward the distal side than the distal end of the balloon 10 such that a distal end portion of the inner shaft 3 functions as the distal tip 8.

In order to enable confirmation of the position of the balloon 10 under X-ray fluoroscopy, one or more radiopaque markers 9 may be provided at a portion of the shaft 2 where the balloon 10 is located in the longitudinal axis direction. The one or more radiopaque markers 9 can be placed, for example, on the inner shaft 3 located inside the balloon 10, and may be positioned at locations corresponding to both ends of the straight tube portion of the balloon 10, or may be positioned at a location corresponding to the center of the straight tube portion of the balloon 10.

The balloon 10 has a longitudinal axis direction and a radial direction, and is formed in a tubular shape having openings on the proximal side and the distal side (see FIGS. 4 and 5). The radial direction of the balloon 10 is a direction orthogonal to the longitudinal axis direction and refers to a direction extending radially from the center of the balloon 10. The balloon 10 also has a circumferential direction as a direction along the outer circumference of the balloon 10 in an inflated state in a cross section of the balloon 10 perpendicular to the longitudinal axis direction.

The balloon 10 includes a straight tube portion 13, a proximal tapered portion 12 located further toward the proximal side than the straight tube portion 13, and a distal tapered portion 14 located further toward the distal side than the straight tube portion 13 with respect to the longitudinal axis direction. The straight tube portion 13 is formed in a substantially cylindrical shape extending in the longitudinal axis direction, and is formed to have the largest length in the radial direction (outer diameter) in the balloon 10. The proximal tapered portion 12 is located on the proximal side of the straight tube portion 13 and connected to the proximal end of the straight tube portion 13. The proximal tapered portion 12 is formed such that the outer diameter thereof decreases with increasing distance from the straight tube portion 13. The distal tapered portion 14 is located on the distal side of the straight tube portion 13 and connected to the distal end of the straight tube portion 13. The distal tapered portion 14 is formed such that the outer diameter thereof decreases with increasing distance from the straight tube portion 13. The balloon 10 may further include a proximal sleeve portion 11 located further toward the proximal side than the proximal tapered portion 12 and a distal sleeve portion 15 located further toward the distal side than the distal tapered portion 14. The proximal sleeve portion 11 is located on the proximal side of the proximal tapered portion 12 and is connected to the proximal end of the proximal tapered portion 12. The proximal sleeve portion 11 is formed in a substantially cylindrical shape. The distal sleeve portion 15 is located on the distal side of the distal tapered portion 14 and is connected to the distal end of the distal tapered portion 14. The distal sleeve portion 15 is formed in a substantially cylindrical shape.

By configuring the balloon 10 as described above, when the balloon 10 is inflated at a stenosed site, the straight tube portion 13 comes into sufficient contact with the stenosed site, making it easier to perform treatment such as expansion of the stenosed site. In addition, since the balloon 10 includes the proximal tapered portion 12 and the distal tapered portion 14, when the balloon 10 is deflated, the outer diameter of a proximal end portion of the balloon 10 and the outer diameter of a distal end portion of the balloon 10 can be reduced so as to reduce the difference in diameter between the shaft 2 and the balloon 10, so that it becomes easier to insert the balloon 10 into a body cavity, a forceps channel of an endoscope, or a delivery catheter such as a guiding catheter.

In the distal portion of the shaft 2, it is preferable that the inner shaft 3 extend to a position further toward the distal side than the distal end of the outer shaft 4, and that the inner shaft 3 extend from the proximal sleeve portion 11 to the distal sleeve portion 15 through the internal space of the balloon 10. It is preferable that the outer surface of the inner shaft 3 be joined to the inner surface of the distal sleeve portion 15 of the balloon 10, and that the outer surface of the outer shaft 4 be joined to the inner surface of the proximal sleeve portion 11 of the balloon 10. With the distal portion of the shaft 2 configured as described above, the balloon inflation fluid can be supplied to the internal space of the balloon 10 through the space between the inner shaft 3 and the outer shaft 4.

The size of the balloon 10 is not particularly limited. The size of the balloon 10 can be appropriately set, for example, with the length of the straight tube portion 13 in the longitudinal axis direction ranging from 4 mm to 400 mm, and the outer diameter of the straight tube portion 13 ranging from 1 mm to 30 mm.

The balloon 10 (particularly a balloon main body 16) may be made of a resin. The resin may be a thermoplastic resin. This makes it easier to manufacture the balloon 10 by molding. Examples of the resin out of which the balloon 10 is made include polyolefin resins such as polyethylene, polypropylene, and ethylene-propylene copolymer; polyester resins such as polyethylene terephthalate and polyester elastomers; polyurethane resins such as polyurethane and polyurethane elastomers; polyphenylene sulfide resin; polyamide resins such as polyamide and polyamide elastomers; fluorine-based resins; silicone resins; and natural rubbers such as latex rubber. Only one of these may be used, or two or more of these may be used in combination. Among these, polyamide resins, polyester resins, and polyurethane resins may be used. In particular, it is preferable to use an elastomer resin from the viewpoint of thinning and flexibility of the balloon 10. Examples of materials suitable for the balloon 10 among polyamide resins include nylon 12, nylon 11, and the like, and nylon 12 may be used because it can be relatively easily molded at the time of blow molding. In addition, from the viewpoint of thinning and flexibility of the balloon 10, polyamide elastomers such as polyether ester amide elastomer and polyamide ether elastomer may be used. Among these, polyether ester amide elastomer may be used from the viewpoint of high yield strength and favorable dimensional stability of the balloon 10.

The balloon 10 includes ridges 21 formed on the outer surface of the straight tube portion 13. Providing the ridges 21 on the outer surface of the straight tube portion 13 gives the balloon 10 a scoring function, and when the balloon 10 is inflated at a stenosed site of a blood vessel, the ridges 21 can bite into the stenosed site that has been calcified, thereby forming cracks in the stenosed site. Therefore, it is possible to expand the stenosed site while suppressing dissection of the blood vessel. Additionally, it is possible to increase the pressure resistance of the balloon 10 and suppress over-inflation of the balloon 10 during pressurization of the balloon 10. Note that, although the balloon 10 can also be used for treatment of a stenosed site or a lesion in a body cavity other than a blood vessel, the following description will mainly focus on the case where the balloon 10 is applied to vascular treatment.

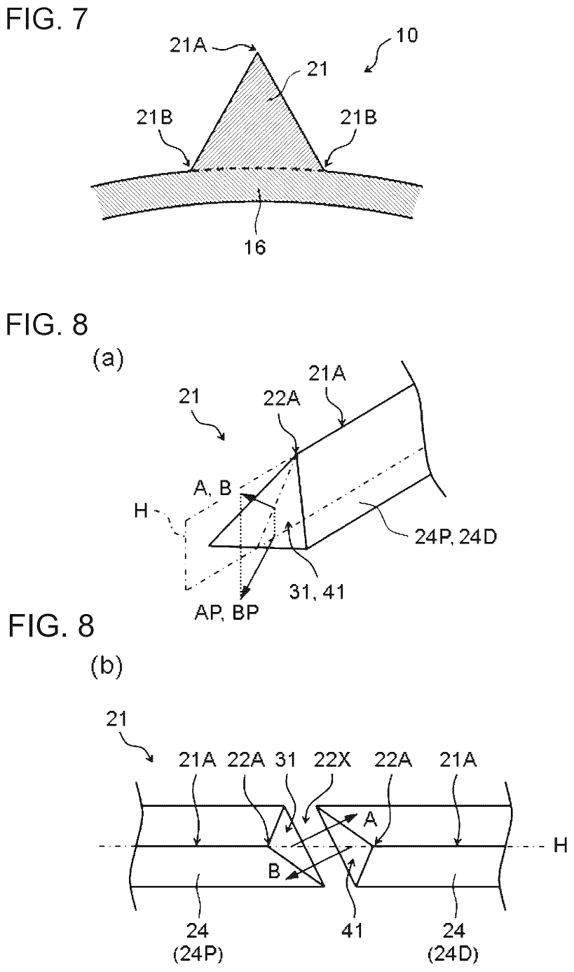

The ridges 21 of the balloon 10 will be described in detail with reference to FIGS. 6 and 7. FIG. 6 illustrates a cross-sectional view of the straight tube portion 13 of the balloon 10 taken perpendicular to the longitudinal axis direction, and FIG. 7 illustrates an enlarged cross-sectional view of one of the ridges 21 of the balloon 10. FIG. 6 illustrates a configuration example of the straight tube portion 13 of the balloon 10, which is illustrated in FIG. 4 and FIG. 5, in a cross section perpendicular to the longitudinal axis direction, and the ridges 21 are provided at three positions in the circumferential direction of the straight tube portion 13.

The straight tube portion 13 of the balloon 10 includes a balloon main body 16 having a cylindrical shape, and the ridges 21 are provided on the outer surface of the balloon main body 16. The ridges 21 are provided so as to protrude in the radial direction outward from the outer surface of the balloon main body 16. In the balloon 10, ridge-present regions 25 and ridge-absent regions 26 are formed on the outer surface of the straight tube portion 13 by providing the ridges 21. The ridge-present regions 25 include portions where notches 22 are formed in the ridges 21, as will be described later. The outer surface of the straight tube portion 13 may be formed flat in the ridge-absent regions 26, and, for example, the outer surface of the straight tube portion 13 may not be formed to be recessed at any portion of the ridge-absent regions 26. This facilitates uniform inflation of the balloon 10 and makes it easier for the ridges 21 to exhibit a desired scoring function. When the outer surface of the straight tube portion 13 is formed flat in the ridge-absent regions 26, each of the ridge-absent regions 26 has an arched shape obtained by bending a flat surface, and no irregularities are formed on the flat surface bent in an arch shape. The irregularities do not include surface roughness that is inevitably formed in manufacturing.

Each of the ridges 21 includes a top portion 21A and a base portion 21B. In each of the ridges 21, the top portion 21A refers to the tip of the ridge 21, that is, the outermost portion of the ridge 21 in the radial direction, and the base portion 21B refers to a boundary between the ridge 21 and the balloon main body 16, that is, the innermost portion of the ridge 21 in the radial direction.

The ridges 21 can be made of, for example, a resin. If the ridges 21 are made of a resin, the balloon 10 including the ridges 21 can be manufactured by resin molding, which facilitates manufacturing. In this case, the ridges 21 and the balloon main body 16 may be made of the same resin, and the ridges 21 and the balloon main body 16 may be integrally molded. The balloon main body 16 may have an inner layer and an outer layer, and in this case, the ridges 21 may be made of the same resin as that of the outer layer of the balloon main body 16. As a result, the ridges 21 are less likely to unintentionally detach from the balloon main body 16. Alternatively, if the resin constituting the ridges 21 and the resin constituting the balloon main body 16 are compatible with each other to some extent, the ridges 21 and the balloon main body 16 may be made of different resins.

The ridges 21 may be made of a metal or may be made of a combination of a metal and a resin. In this case, it is preferable that portions of the ridges 21 including the top portions 21A be made of a metal. This makes it easier for the ridges 21 to form cracks in a stenosed site or cut open the stenosed site when the balloon 10 is inflated. For example, the entirety of the ridges 21 may be made of a metal, or portions of the ridges 21 including the base portions 21B may be made of a resin while portions of the ridges 21 including the top portions 21A may be made of a metal. Therefore, the ridges 21 may be made of a resin, a metal, or a combination of these.

In the straight tube portion 13, the balloon main body 16 is defined as a portion having a tubular shape. A portion of the straight tube portion 13 excluding the ridges 21 protruding outward in the radial direction corresponds to the balloon main body 16. The balloon main body 16 can be regarded as having a cylindrical outer surface. Therefore, in a cross section of the straight tube portion 13 perpendicular to the longitudinal axis direction, the outer shape of the balloon main body 16 is formed in a substantially circular shape, so that the balloon main body 16 and the ridges 21 can be distinguished from each other. The ridge-present regions 25 are constituted by the balloon main body 16 and the ridges 21, and the ridge-absent regions 26 are constituted by the balloon main body 16.

The ridges 21 are provided on the outer surface of the straight tube portion 13 in such a manner as to extend in a ridge-like manner. The ridges 21 extend substantially parallel to the longitudinal axis direction of the balloon 10. Therefore, each of the ridges 21 has an extension direction substantially parallel to the longitudinal axis direction of the balloon 10, and a width direction corresponding to the circumferential direction of the balloon 10.

The plurality of ridges 21 are provided at different positions on the straight tube portion 13 of the balloon 10 in the circumferential direction. That is, the ridges 21 are provided at a plurality of positions in the circumferential direction of the balloon 10. In this case, the ridges 21 may be arranged at substantially equal intervals in the circumferential direction of the straight tube portion 13 of the balloon 10. As a result, when the balloon 10 is inflated, it is possible to form cracks in a plurality of portions of the stenosed site. In the circumferential direction of the balloon 10, the ridges 21 may be provided at two or more positions, at three or more positions, and may be at twelve or fewer positions, at ten or fewer positions, or at eight or fewer positions. In this case, it is preferable that the interval between the ridges 21 in the circumferential direction be longer than the length of one of the ridges 21 in the circumferential direction.

The plurality of ridges 21 may be provided at substantially the same position in the longitudinal axis direction. That is, the proximal ends of the plurality of ridges 21 may be located at substantially the same position in the longitudinal axis direction, and the distal ends of the plurality of ridges 21 may be located at substantially the same position in the longitudinal axis direction.

The cross-sectional shape of each of the ridges 21 is not particularly limited. For example, the shape of each of the ridges 21 in a cross section of the straight tube portion 13 perpendicular to the longitudinal axis direction may be a polygonal shape, such as a triangular shape or a rectangular shape, a partial shape of a circle, such as a semicircular shape or a sector shape, a wedge shape, a protruding shape, a spindle shape, an irregular shape, or the like. The polygonal shape includes a rounded polygonal shape with rounded corners and a polygonal shape with at least partially curved sides, in addition to a polygonal shape with clearly defined corner vertices and straight sides. Note that each of the ridges 21 may be formed such that the width thereof gradually decreases toward the top portion 21A.

In a cross section of the straight tube portion 13 perpendicular to the longitudinal axis direction, the height of each of the ridges 21 may be 0.2 times or more the width (maximum width) of the ridge 21. If the ridges 21 are formed in this manner, when the balloon 10 is inflated at a stenosed site, the ridges 21 easily bite into the stenosed site, and the scoring function provided by the ridges 21 can be enhanced. Each of the ridges 21 may be formed so as to be widest at the base portion 21B, so that the ridge 21 is stably provided on the outer surface of the balloon main body 16. The height of each of the ridges 21 may be 0.4 times or more the width of the ridge 21, or 0.7 times or more the width of the ridge 21. On the other hand, the height of each of the ridges 21 may be 2.0 times or less the width of the ridge 21, 1.8 times or less the width of the ridge 21, or 1.5 times or less the width of the ridge 21. This makes it easier to ensure the flexibility of the balloon 10 at the portions of the balloon 10 where the ridges 21 are present.

In the straight tube portion 13, the thickness of the portion where the ridges 21 are provided, that is, the thickness of the ridge-present regions 25, may be formed to be thicker than the thickness of the portion where the ridges 21 are not provided, that is, the thickness of the ridge-absent regions 26. This can enhance the scoring function provided by the ridges 21. The thickness (maximum thickness) of the ridge-present regions 25 may be 1.5 times or more, 2.0 times or more, or 2.5 times or more the thickness (maximum thickness) of the ridge-absent regions 26. The upper limit of the thickness of the ridge-present regions 25 is not particularly limited and may be, for example, 30 times or less, 20 times or less, or 10 times or less the thickness of the ridge-absent regions 26.

In the balloon 10, the ridges 21 may be provided in an area of 60% or more of the straight tube portion 13 in the longitudinal axis direction, in an area of 70% or more, or in an area of 80% or more. This enables formation of cracks in a wide area of a stenosed site when the balloon 10 is inflated. The ridges 21 may be provided in an area of 90% or more of the straight tube portion 13 in the longitudinal axis direction, may be provided over substantially the entire straight tube portion 13 in the longitudinal axis direction, or may be provided on the outer surface of the proximal tapered portion 12 and/or the proximal tapered portion 14. The ridges 21 may also be provided on the outer surface of the proximal sleeve portion 11 and/or the distal sleeve portion 15.

The balloon 10 may include an inner ridge (not illustrated) provided on the inner surface of the balloon 10 in such a manner as to protrude toward the inner side in the radial direction. The inner ridge and at least one of the ridges 21 may be arranged at the same position in the longitudinal axis direction of the balloon 10 and in the circumferential direction of the balloon 10, and they may be integrally formed, so that a portion of the balloon 10 may be formed to be thick.

The balloon 10 provided with the ridges 21 tends to have high rigidity at the portions thereof where the ridges 21 are provided. Therefore, the balloon 10 provided with the ridges 21 is more likely to have reduced flexibility in the longitudinal axis direction, compared with the balloon 10 without the ridges 21. For example, in a shunt that is formed during hemodialysis, a blood vessel bends significantly at an arteriovenous anastomosis site. When passing a balloon through such a location, there is a risk that the balloon with ridges may have difficulty passing through the arteriovenous anastomosis site. In the case of a lower limb balloon, the balloon is inserted into an iliac artery during treatment. However, at the bifurcation where the left and right iliac arteries branch from the abdominal aorta, a blood vessel bends significantly. Therefore, if the lower limb balloon is provided with ridges, there is a risk that it may be difficult to pass the balloon from one of the left and right iliac arteries to the other. In particular, since a lower limb balloon is long, there is a higher risk that the balloon cannot pass through a location where a blood vessel bends significantly. Accordingly, as illustrated in FIGS. 4 and 5, the notches 22 are formed in the ridges 21 of the balloon 10. By forming the notches 22 in the ridges 21, the flexibility of the balloon 10 in the longitudinal axis direction can be increased.

The notches 22 may be formed in each of the ridges 21. This makes it possible to increase the flexibility of the balloon 10 regardless of the direction in which the balloon 10 is bent.

The number of the notches 22 formed in each of the ridges 21 is not particularly limited as long as it is one or more. However, from the viewpoint of increasing the flexibility of the balloon 10, the number of the notches 22 formed in each of the ridges 21 may be two or more, or three or more. On the other hand, from the viewpoint of ensuring the scoring function of the balloon 10, the number of the notches 22 formed in each of the ridges 21 may be 20 or fewer, 16 or fewer, 12 or fewer, or 8 or fewer.

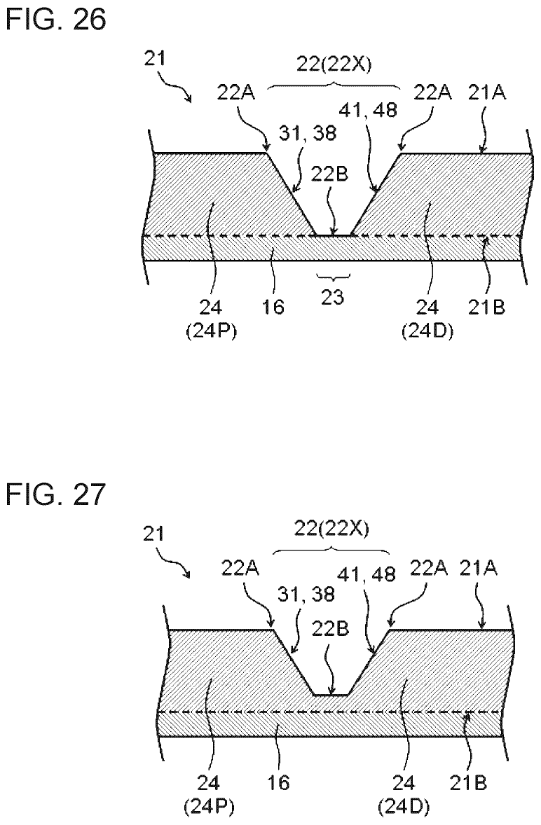

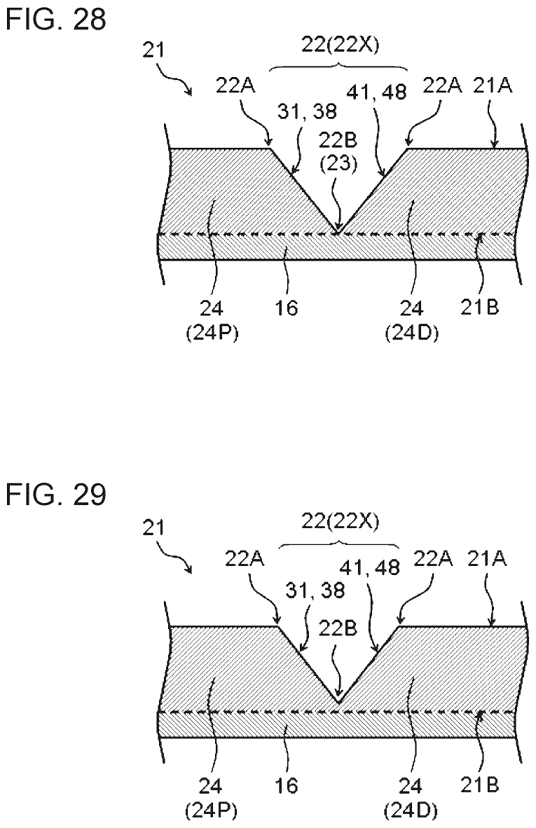

Each of the notches 22 may be formed in such a manner that it partially cuts off the top portion 21A of the corresponding ridge 21, which extends in the longitudinal axis direction. FIGS. 26 to 29 illustrate cross-sectional views of a specific notch 22X, which will be described later, taken along the longitudinal axis direction and passing through the top portion 21A of the ridge 21. However, as illustrated in FIGS. 26 and 28, each of the notches 22 may be formed in such a manner as to extend from the top portion 21A to the base portion 21B of the corresponding ridge 21. Alternatively, as illustrated in FIGS. 27 and 29, each of the notches 22 may be formed in such a manner as to extend from the top portion 21A of the corresponding ridge 21 to an intermediate position between the top portion 21A and the base portion 21B of the ridge 21. In the former case, the depth of each of the notches 22 is equal to the height of the corresponding ridge 21. In the latter case, the depth of each of the notches 22 is formed to be shorter than the height of the corresponding ridge 21. FIGS. 26 to 29 each illustrate a cross-sectional view of one of the ridges 21 including one of the notches 22, taken by a virtual plane passing through the top portion 21A of the ridge 21 and formed by the longitudinal axis direction and the radial direction of the balloon 10.

Each of the notches 22 has a bottom portion 22B and top portions 22A. The bottom portion 22B corresponds to the innermost portion of the notch 22 in the radial direction, and the top portions 22A correspond to the outermost portions of the notch 22 in the radial direction. Each of the top portions 22A of the notch 22 coincides with the top portion 21A of the ridge 21. The length in the radial direction of the notch 22 from each of the top portions 22A to the bottom portion 22B is the depth of the notch 22.

Each of the ridges 21 is divided into a plurality of ridge segments 24 by the notches 22. To be more specific, with the bottom portion 22B of the notch 22 as a boundary, it is divided into the ridge segment 24 on the proximal side and the ridge segment 24 on the distal side. Hereinafter, the ridge segment 24 adjacent to the notch 22 on the proximal side will be referred to as a proximal ridge segment 24P, and the ridge segment 24 adjacent to the notch 22 on the distal side will be referred to as a distal ridge segment 24D.

As illustrated in FIGS. 26 and 27, when the bottom portion 22B of one of the notches 22 is formed to have a predetermined length in the longitudinal axis direction, the proximal ridge segment 24P is formed on the proximal side of the proximal end of the bottom portion 22B of the notch 22, and the distal ridge segment 24D is formed on the distal side of the distal end of the bottom portion 22B of the notch 22. That is, the proximal end of the bottom portion 22B of the notch 22 serves as a distal boundary of the proximal ridge segment 24P, and the distal end of the bottom portion 22B of the notch 22 serves as a proximal boundary of the distal ridge segment 24D. When the notches 22 are formed so as to extend from the top portions 21A to the base portions 21B of the ridges 21 as illustrated in FIGS. 26 and 28, interrupted portions 23 of the ridges 21 are formed by the notches 22, and the ridge segments 24 are arranged on the proximal sides and the distal sides of the interrupted portions 23. In this case, the ridges 21 are formed by alternately arranging the ridge segments 24 and the interrupted portions 23 in the longitudinal axis direction. The proximal ridge segments 24P and the distal ridge segments 24D adjacent to the notches 22 may be provided to be spaced apart from each other in the longitudinal axis direction as illustrated in FIGS. 26 and 27, or may be provided to be in contact with each other in the longitudinal axis direction as illustrated in FIGS. 28 and 29.

The length of each of the notches 22 in the longitudinal axis direction may be shorter than the length of each of the ridge segments 24 in the longitudinal axis direction. Specifically, in each of the ridges 21, the length of the notch 22 in the longitudinal axis direction (when the plurality of notches 22 are provided, the length of each of the notches 22 in the longitudinal axis direction) may be shorter than the length of any of the ridge segments 24 in the longitudinal axis direction. In addition, in each of the ridges 21, the length of the notch 22 in the longitudinal axis direction (when the plurality of notches 22 are provided, the length of each of the notches 22 in the longitudinal axis direction) may be 0.5 times or less, 0.3 times or less, or 0.2 times or less the average value of the lengths of the ridge segments 24 in the longitudinal axis direction. This makes it easier to ensure the scoring function provided by the ridges 21. In a cross section taken along the longitudinal axis direction and passing through the top portion 21A of each of the ridges 21, the length of each of the notches 22 in the longitudinal axis direction refers to a distance between a top portion of the ridge segment 24 located on the proximal side of the notch 22 and a top portion of the ridge segment 24 located on the distal side of the notch 22, that is, a distance between the distal end of the top portion of the proximal ridge segment 24P and the proximal end of the top portion of the distal ridge segment 24D.

In each of the ridges 21, the total length of the notches 22 in the longitudinal axis direction may be 20% or less, 15% or less, or 10% or less of the length of the ridge 21 in the longitudinal axis direction. This makes it easier to ensure the scoring function provided by the ridges 21. The length of each of the ridges 21 in the longitudinal axis direction is determined as follows. Among the plurality of ridge segments 24 constituting each ridge 21, the proximal end of the ridge segment 24 on the most proximal side is the proximal end of the ridge 21. The distal end of the ridge segment 24 on the most distal side is the distal end of the ridge 21. The length in the longitudinal axis direction from the proximal end to the distal end of the ridge 21 is the length of the ridge 21 in the longitudinal axis direction.

In each of the notches 22, the length of the notch 22 in the longitudinal axis direction may be 0.2 times or more, 0.3 times or more, or 0.5 times or more the depth of the notch 22. This makes it easier to increase the flexibility of the straight tube portion 13 of the balloon 10 in the longitudinal axis direction. In each of the notches 22, the length of the notch 22 in the longitudinal axis direction may be 5 times or less, 3 times or less, or 2 times or less the depth of the notch 22. This makes it easier to ensure the scoring function of the balloon 10.

It is preferable that the length of a portion of each of the notches 22 where the bottom portion 22B extends parallel to the longitudinal axis direction not be formed to an excessive length compared with the length of the notch 22 in the longitudinal axis direction (the distance between the top portion of the ridge segment 24 located on the proximal side of the notch 22 and the top portion of the ridge segment 24 located on the distal side of the notch 22). In each of the notches 22, the length of the portion of each of the notches 22 where the bottom portion 22B extends parallel to the longitudinal axis direction may be 0.5 times or less, 0.3 times or less, or 0.2 times or less the length of the notch 22 in the longitudinal axis direction. This facilitates smooth bending of the balloon 10 at the notches 22 and makes it easier to ensure the scoring function provided by the ridges 21. The bottom portion 22B of each of the notches 22 does not necessarily include a portion extending parallel to the longitudinal axis direction. As illustrated in FIGS. 26 and 28, in the case where one of the notches 22 is formed in such a manner as to extend from the top portion 21A to the base portion 21B of the corresponding ridge 21, the length of the interrupted portion 23 of the ridge 21 in the longitudinal axis direction (the length of the interrupted portion 23 on the outer surface of the balloon main body 16 in the longitudinal axis direction) corresponds to the length of the bottom portion 22B of the notch 22 in the longitudinal axis direction.

The notches 22 may be formed at any positions in the longitudinal axis direction of the ridges 21, which are provided on the straight tube portion 13. For example, as illustrated in FIGS. 4 and 5, in the straight tube portion 13, when one of the ridges 21 is equally divided into three sections in the longitudinal axis direction, that is, a proximal section 17, an intermediate section 18, and a distal section 19, the notches 22 may be provided in any of the proximal section 17, the intermediate section 18, or the distal section 19.

In one embodiment, it is preferable that the notches 22 be provided at least in the distal sections 19. By forming the notches 22 in the ridges 21 in the manner described above, it is possible to increase the flexibility of a distal portion of the balloon 10 (specifically, a portion of the balloon 10 corresponding to the distal sections 19 of the ridges 21 in the longitudinal axis direction). In this case, the notches 22 may be provided in the distal sections 19 of the ridges 21 provided on the straight tube portion 13.

It is also preferable that the notches 22 be provided in the proximal sections 17 of the ridges 21. This can increase the flexibility of a proximal portion of the balloon 10 (specifically, a portion of the balloon 10 corresponding to the proximal sections 17 of the ridges 21 in the longitudinal axis direction), and when the balloon 10 is pulled back and passed through a curved portion after treatment using the balloon 10, the maneuverability of the balloon 10 through the curved portion can be improved. In this case, the notches 22 may be provided in the proximal sections 17 of the ridges 21 provided on the straight tube portion 13.

The proximal section 17, the intermediate section 18, and the distal section 19 of each of the ridges 21 are defined as follows. In each of the ridges 21, when the length in the longitudinal axis direction from the proximal end to the distal end of the ridge 21 is L, the ridge 21 is divided into three sections, each having a length of L/3. The section on the most proximal side is defined as the proximal section 17, the section on the most distal side is defined as the distal section 19, and the section between the proximal section 17 and the distal section 19 is defined as the intermediate section 18. The positions of the notches 22 in the longitudinal axis direction of the ridges 21, that is, whether each of the notches 22 is located in the proximal section 17, the intermediate section 18, or the distal section 19 of the ridge 21, are determined based on the position of the bottom portion 22B of the notch 22 in a cross section taken along the longitudinal axis direction and passing through the top portion 21A of the corresponding ridge 21 (to be specific, a cross section taken along the longitudinal axis direction and the radial direction and passing through the top portion 21A of the ridge 21). When the bottom portion 22B of one of the notches 22 is formed to have a predetermined length in the longitudinal axis direction, a midpoint of the bottom portion 22B of the notch 22 in the longitudinal axis direction is defined as the position of the notch 22 in the corresponding ridge 21 in the longitudinal axis direction. When the bottom portion 22B of the notch 22 is positioned exactly at the boundary between the proximal section 17 and the intermediate section 18 or at the boundary between the intermediate section 18 and the distal section 19, the notch 22 is determined to be present in both sections but not to belong to either section.

The notches 22 may not be provided in the intermediate sections 18 of the ridges 21. If the ridges 21 are formed in the manner described above, it becomes easier to impart a high scoring function to the balloon 10 while increasing the flexibility of the balloon 10. On the other hand, in order to further increase the flexibility of the balloon 10, the notches 22 may also be provided in the intermediate sections 18 of the ridges 21. For example, since the balloon 10 for lower limbs is long in the longitudinal axis direction, by providing the notches 22 also in the intermediate sections 18, even in the balloon 10 that is long in the longitudinal axis direction, the flexibility can be ensured over the entire balloon 10 in the longitudinal axis direction.

In the balloon 10, when the ridges 21 are further provided on at least one of the proximal sleeve portion 11, the proximal tapered portion 12, the distal tapered portion 14, and the distal sleeve portion 15, the notches 22 may also be provided on the ridges 21 on the proximal sleeve portion 11, the proximal tapered portion 12, the distal tapered portion 14, or the distal sleeve portion 15. By providing the notches 22 in this manner, the flexibility of the balloon 10 can be enhanced also in a portion other than the straight tube portion 13.

The ridges 21 are provided with a specific notch 22X that satisfies Requirement A or Requirement B below, the specific notch 22X being provided as some or all of the notches 22 described above. In the balloon 10 according to the first embodiment illustrated in FIG. 4, the ridges 21 are provided with the specific notch 22X satisfying Requirement A below, and in the balloon 10 according to the second embodiment illustrated in FIG. 5, the ridges 21 are provided with the specific notch 22X satisfying Requirement B below.

(Requirement A) A distal surface of the proximal ridge segment 24P adjacent to the specific notch 22X on the proximal side has a normal vector A on a line of intersection with a virtual plane H formed by the longitudinal axis direction and the radial direction and passing through the top portion 21A of the corresponding ridge 21, the normal vector A being oriented toward the distal side on one side of the virtual plane H. A proximal surface of the distal ridge segment 24D adjacent to the distal side of the specific notch 22X has a normal vector B on a line of intersection with the virtual plane H, the normal vector B being oriented toward the proximal side on the other side of the virtual plane H.

(Requirement B) In a plan view from the top portion 21A of the corresponding ridge 21, a distal end edge of the proximal ridge segment 24P adjacent to the specific notch 22X on the proximal side has a protruding shape protruding toward the distal side, and a proximal end edge of the distal ridge segment 24D adjacent to the specific notch 22X on the distal side has a protruding shape protruding toward the proximal side.

The configuration of one of the ridges 21 provided on the balloon 10 of the first embodiment, in particular, the configuration of end portions of the ridge segments 24 that are adjacent to the specific notch 22X satisfying Requirement A, will be described with reference to FIGS. 8 to 15. FIGS. 8 to 15 illustrate various configuration examples of the end portions of the ridge segments 24 adjacent to the specific notch 22X satisfying Requirement A. Each of FIG. 8(a) to 15(a) is a perspective view of a distal end portion of the proximal ridge segment 24P as viewed from the distal side or a perspective view of a proximal end portion of the distal ridge segment 24D as viewed from the proximal side. Each of FIG. 8(b) to 15(b) is a plan view of the distal end portion of the proximal ridge segment 24P and the proximal end portion of the distal ridge segment 24D as viewed from the top portion 21A of the corresponding ridge 21. Note that the end portion of the ridge segment 24 refers to the distal end portion of the proximal ridge segment 24P and/or the proximal end portion of the distal ridge segment 24D adjacent to the specific notch 22X.

In each of the ridges 21 of the balloon 10 according to the first embodiment, the distal end portion of the proximal ridge segment 24P and the proximal end portion of the distal ridge segment 24D adjacent to the specific notch 22X are formed as follows. A distal surface 31 of the proximal ridge segment 24P has the normal vector A on the line of intersection with the virtual plane H formed by the longitudinal axis direction and the radial direction and passing through the top portion 21A of the ridge 21, the normal vector A being oriented toward the distal side on one side of the virtual plane H. A proximal surface 41 of the distal ridge segment 24D has the normal vector B on the line of intersection with the virtual plane H, the normal vector B being oriented toward the proximal side on the other side of the virtual plane H. As illustrated in FIGS. 8(b) to 15(b), the orientation direction of the normal vector A of the distal surface 31 of the proximal ridge segment 24P and the orientation direction of the normal vector B of the proximal surface 41 of the distal ridge segment 24D are defined in a state where the proximal ridge segment 24P and the distal ridge segment 24D are arranged with the specific notch 22X interposed therebetween.

Since the distal end portion of the proximal ridge segment 24P and the proximal end portion of the distal ridge segment 24D are formed as described above, in a plan view as viewed from the top portion 21A of the ridge 21, at least a portion (specifically, at least a portion including a portion intersecting the virtual plane H) of the distal surface 31 of the proximal ridge segment 24P and at least a portion (specifically, at least a portion including a portion intersecting the virtual plane H) of the proximal surface 41 of the distal ridge segment 24D face each other and are formed so as to extend obliquely with respect to the longitudinal axis direction. Therefore, if the balloon 10 is bent with the specific notch 22X on the inner side, when the distal surface 31 of the proximal ridge segment 24P and the proximal surface 41 of the distal ridge segment 24D, both adjacent to the specific notch 22X, come into contact with each other, the distal end portion of the proximal ridge segment 24P and the proximal end portion of the distal ridge segment 24D are more easily displaced from each other in the width direction of the ridge 21. This makes it easier to greatly bend the balloon 10.

The distal surface 31 of the proximal ridge segment 24P refers to a portion of the proximal ridge segment 24P that faces the distal side. On the distal surface 31 of the proximal ridge segment 24P, the normal vector A on the line of intersection with the virtual plane H formed by the longitudinal axis direction and the radial direction and passing through the top portion 21A of the ridge 21 is oriented toward the distal side on one side (first side) of the virtual plane H. The normal vector A is oriented, toward the distal side, from the other side (second side) toward the one side (first side) in the circumferential direction of the balloon 10. The normal vector A may be oriented parallel to the outer surface of the balloon main body 16, that is, such that the position in the radial direction does not change, may extend radially outward toward the distal side, or may be oriented radially inward toward the distal side. It is only necessary that the distal surface 31 of the proximal ridge segment 24P be formed such that such a normal vector A is present at least in part along the line of intersection with the virtual plane H. However, it is preferable that the distal surface 31 of the proximal ridge segment 24P be formed to have such a normal vector A at the corresponding top portion 22A of the notch 22X on the line of intersection with the virtual plane H, and it is more preferable that the distal surface 31 of the proximal ridge segment 24P be formed to have such a normal vector A along the entire line of intersection with the virtual plane H.

The proximal surface 41 of the distal ridge segment 24D refers to a portion of the distal ridge segment 24D that faces the proximal side. On the proximal surface 41 of the distal ridge segment 24D, the normal vector B on the line of intersection with the virtual plane H passing through the top portion 21A of the ridge 21 and formed by the longitudinal axis direction and the radial direction is oriented toward the proximal side on the other side of the virtual plane H (the side opposite to the normal vector A across the virtual plane H; the second side). The normal vector B is oriented, toward the proximal side, from the one side (first side) toward the other side (second side) in the circumferential direction of the balloon 10. The normal vector B may be oriented parallel to the outer surface of the balloon main body 16 (i.e., such that the position in the radial direction does not change), may be oriented radially outward toward the proximal side, or may be oriented radially inward toward the proximal side. It is only necessary that the proximal surface 41 of the distal ridge segment 24D be formed such that such a normal vector B is present at least in part on the line of intersection with the virtual plane H, but it is preferable that the proximal surface 41 of the distal ridge segment 24D be formed to have such a normal vector B at the corresponding top portion 22A of the notch 22X on the line of intersection with the virtual plane H, and it is more preferable that the proximal surface 41 of the distal ridge segment 24D be formed to have such a normal vector B along the entire line of intersection with the virtual plane H.

The distal surface 31 of the proximal ridge segment 24P and the proximal surface 41 of the distal ridge segment 24D may each be formed in a flat shape, in a curved shape, or in a combination thereof. The shape of the distal surface 31 of the proximal ridge segment 24P may be the same as or different from the shape of the proximal surface 41 of the distal ridge segment 24D. Furthermore, the shapes of the distal surfaces 31 of the plurality of proximal ridge segments 24P may be the same as or different from each other, and the shapes of the proximal surfaces 41 of the plurality of distal ridge segments 24D may be the same as or different from each other.

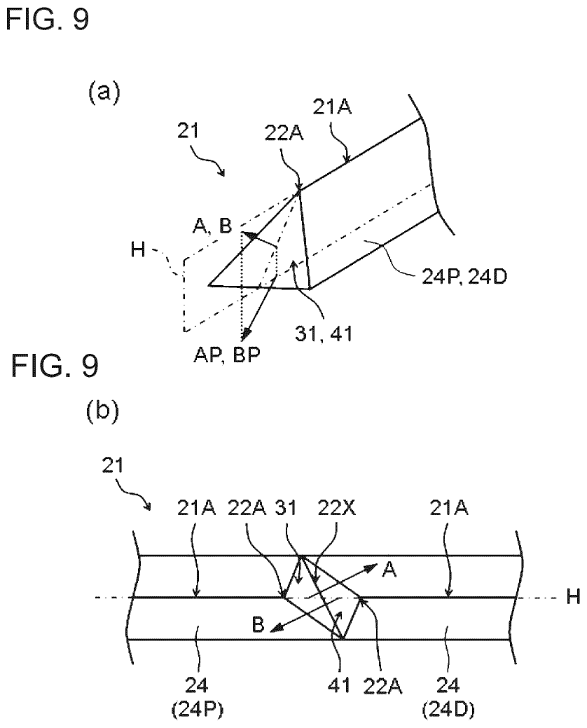

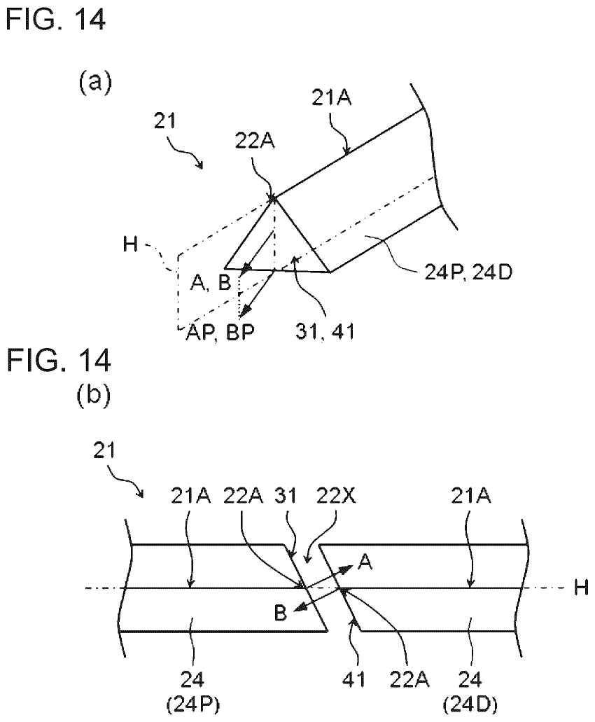

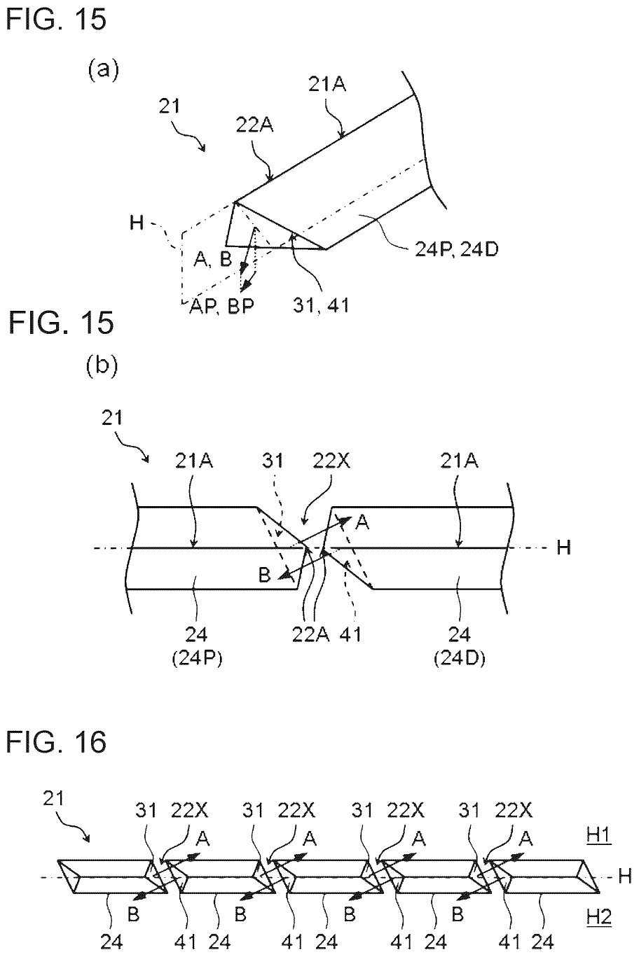

For example, as illustrated in FIG. 8, FIG. 9, FIG. 14, and FIG. 15, the distal surface 31 of the proximal ridge segment 24P may be composed of one surface, and the proximal surface 41 of the distal ridge segment 24D may be composed of one plane. In this case, the normal vector of the entire distal surface 31 of the proximal ridge segment 24P coincides with the normal vector A, and the normal vector of the entire proximal surface 41 of the distal ridge segment 24D coincides with the normal vector B.

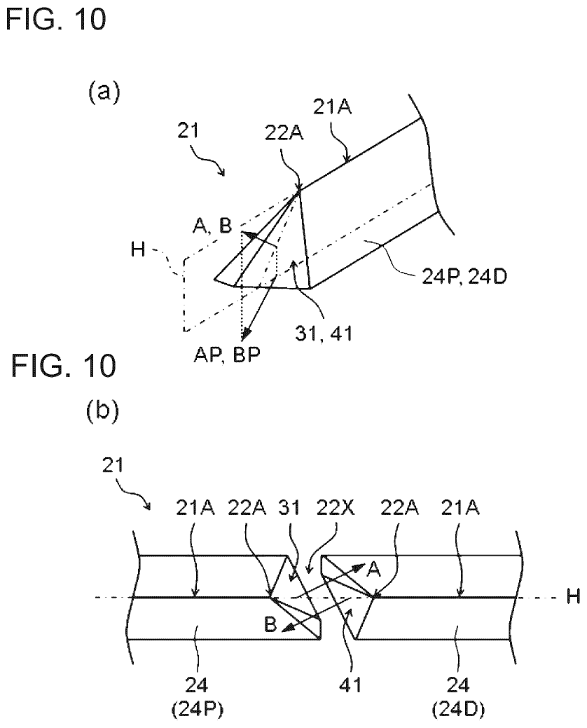

As illustrated in FIGS. 10 to 13, the distal surface 31 of the proximal ridge segment 24P may be composed of two or more flat surfaces, and the proximal surface 41 of the distal ridge segment 24D may be composed of two or more flat surfaces. In this case, at least one of the planes constituting the distal surface 31 of the proximal ridge segment 24P, the at least one flat surface intersecting the virtual plane H, has the normal vector A described above, and at least one of the planes constituting the proximal surface 41 of the distal ridge segment 24D, the at least one flat surface intersecting the virtual plane H, has the normal vector B described above.

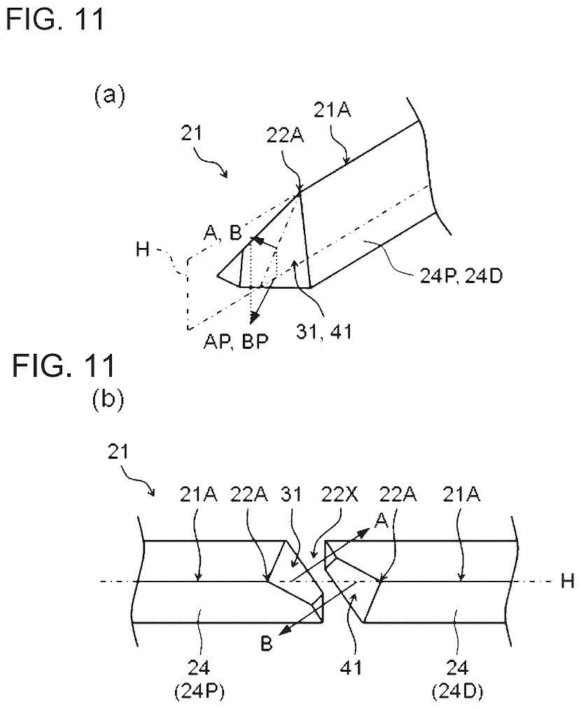

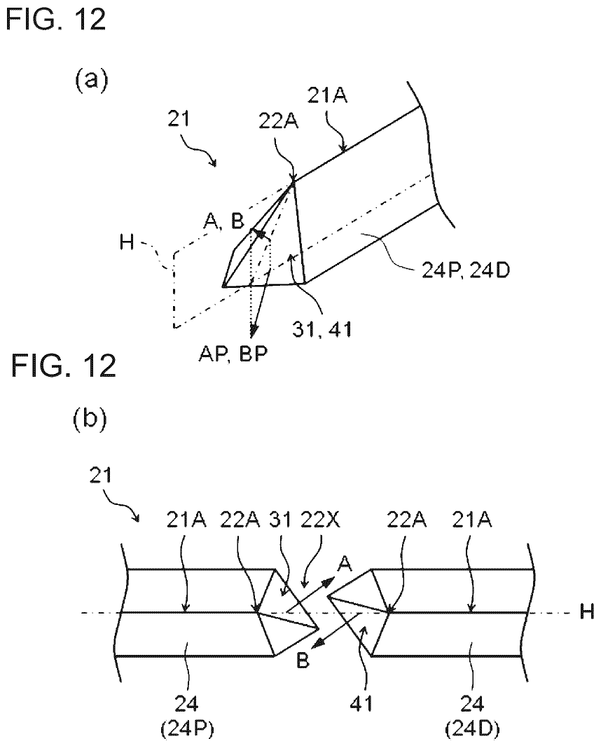

As illustrated in FIGS. 10 and 11, among the flat surfaces constituting the distal surface 31 of the proximal ridge segment 24P, the flat surface that does not intersect the virtual plane H may be formed such that the normal vector is oriented parallel to the virtual plane H, or may be formed such that the normal vector is oriented in a direction opposite to the normal vector A in the circumferential direction toward the distal side, as illustrated in FIG. 12. In addition, although not illustrated in the drawings, the distal surface 31 of the proximal ridge segment 24P may have a flat surface having a normal vector being oriented in a direction different from the normal vector A and in the same direction as the normal vector A in the circumferential direction toward the distal side.

Among the flat surfaces constituting the distal surface 31 of the proximal ridge segment 24P, the flat surface that does not have the normal vector A may be formed so as to include the corresponding top portion 22A of the specific notch 22X as illustrated in FIGS. 10 and 12, or may be formed so as not to include the corresponding top portion 22A of the specific notch 22X as illustrated in FIG. 11.

Among the flat surfaces constituting the proximal surface 41 of the distal ridge segment 24D, the flat surface that does not intersect the virtual plane H may be formed such that the normal vector is oriented parallel to the virtual plane H as illustrated in FIGS. 10 and 11, or may be formed such that the normal vector is oriented in a direction opposite to the normal vector B in the circumferential direction toward the proximal side as illustrated in FIG. 12. Furthermore, although not illustrated in the drawings, the proximal surface 41 of the distal ridge segment 24D may have a flat surface having a normal vector oriented in a direction different from the normal vector B and in the same direction as the normal vector B in the circumferential direction toward the proximal side.

Among the flat surfaces constituting the proximal surface 41 of the distal ridge segment 24D, the flat surface not having the normal vector B may be formed so as to include the corresponding top portion 22A of the specific notch 22X as illustrated in FIGS. 10 and 12, or may be formed so as not to include the corresponding top portion 22A of the specific notch 22X as illustrated in FIG. 11.

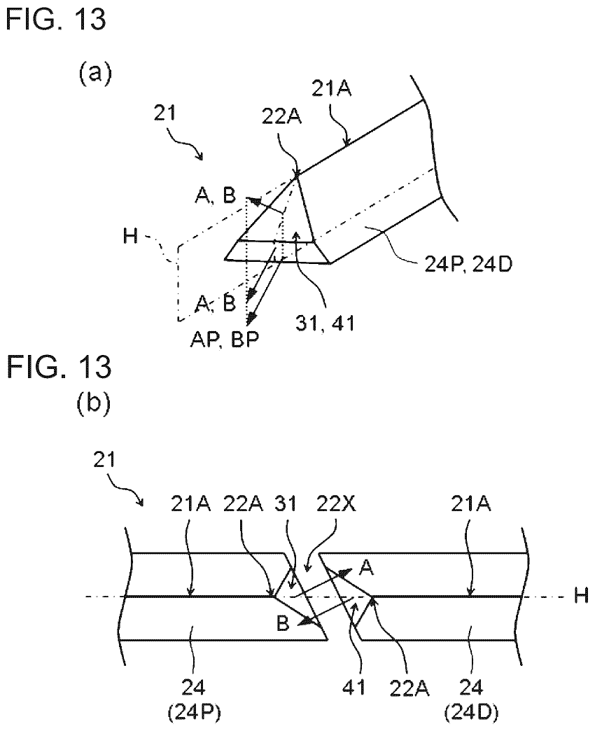

In FIG. 13, the distal surface 31 of the proximal ridge segment 24P is composed of two or more flat surfaces, each intersecting the virtual plane H and having the normal vector A described above, and the proximal surface 41 of the distal ridge segment 24D is composed of two or more flat surfaces, each intersecting the virtual plane H and having the normal vector B described above. As described above, the distal surface 31 of the proximal ridge segment 24P may be composed of two or more flat surfaces intersecting the virtual plane H, and for each of the flat surfaces, the normal vector on the line of intersection with the virtual plane H may be formed so as to be oriented toward the distal side on one side of the virtual plane H. The proximal surface 41 of the distal ridge segment 24D may be composed of two or more flat surfaces intersecting the virtual plane H, and for each of the flat surfaces, the normal vector on the line of intersection with the virtual plane H may be formed so as to be oriented toward the proximal side on the other side of the virtual plane H.

Although not illustrated in FIGS. 8 to 15, the distal surface 31 of the proximal ridge segment 24P may include a curved surface, and the proximal surface 41 of the distal ridge segment 24D may include a curved surface. It is preferable that the distal surface 31 of the proximal ridge segment 24P be formed such that the area of a region formed such that the normal vector is oriented toward the distal side and from the other side toward the one side in the circumferential direction may be 50% or more of the entire area of the distal surface 31, 60% or more, or 70% or more. It is preferable that the proximal surface 41 of the distal ridge segment 24D be formed such that the area of a region formed such that the normal vector is oriented toward the proximal side and from one side toward the other side in the circumferential direction may be 50% or more of the entire area of the proximal surface 41, 60% or more, or 70% or more.

In a plan view as viewed from the top portion 21A of the ridge 21, the angle formed between the normal vector A and the longitudinal axis direction toward the distal side, that is, the angle formed between a projection vector AP of the normal vector A onto the outer surface of the balloon main body 16 and the longitudinal axis direction toward the distal side, may be 20° or more, or 30° or more, and may be 70° or less, or 60° or less. In a plan view as viewed from the top portion 21A of the ridge 21, the angle formed between the normal vector B and the longitudinal axis direction toward the proximal side, that is, the angle formed between a projection vector BP of the normal vector B onto the outer surface of the balloon main body 16 and the longitudinal axis direction toward the proximal side, may be 20° or more, or 30° or more, and may be 70° or less, or 60° or less. When the distal surface 31 of the proximal ridge segment 24P and the proximal surface 41 of the distal ridge segment 24D are formed as described above, if the balloon 10 is bent with the specific notch 22X on the inner side and the distal surface 31 of the proximal ridge segment 24P and the proximal surface 41 of the distal ridge segment 24D come into contact with each other, the distal end portion of the proximal ridge segment 24P and the proximal end portion of the distal ridge segment 24D are more easily displaced from each other in the width direction of the ridge 21. This makes it easier to greatly bend the balloon 10. Note that the angle formed between the projection vector AP and the longitudinal axis direction toward the distal side and the angle formed between the projection vector BP and the longitudinal axis direction toward the proximal side are each in the range of more than 0° and less than 90°.

It is preferable that the distal surface 31 of the proximal ridge segment 24P and the proximal surface 41 of the distal ridge segment 24D be closest to each other in the longitudinal axis direction at least on the line of intersection with the virtual plane H. That is, it is preferable that the separation distance in the longitudinal axis direction between the distal surface 31 of the proximal ridge segment 24P and the proximal surface 41 of the distal ridge segment 24D be shortest at least on the line of intersection with the virtual plane H. A distal portion of the proximal ridge segment 24P and a proximal portion of the distal ridge segment 24D illustrated in FIGS. 8 to 15 are formed in this manner. This makes it easier to greatly bend the balloon 10 with the specific notch 22X on the inner side. It is preferable that the distal surface 31 of the proximal ridge segment 24P and the proximal surface 41 of the distal ridge segment 24D be closest to each other in the longitudinal axis direction at least on the line of intersection with the virtual plane H in any vertical cross section in the radial direction. Note that the distal surface 31 of the proximal ridge segment 24P and the proximal surface 41 of the distal ridge segment 24D may be closest to each other in the longitudinal axis direction not only on the line of intersection with the virtual plane H but also in other portions.

It is preferable that the separation distance between the distal surface 31 of the proximal ridge segment 24P and the proximal surface 41 of the distal ridge segment 24D in the longitudinal axis direction be formed so as to remain unchanged or to increase with increasing distance from the virtual plane H, and it is more preferable that the separation distance be formed so as to increase at least in part with increasing distance from the virtual plane H. The distal portion of the proximal ridge segment 24P and the proximal portion of the distal ridge segment 24D illustrated in FIGS. 8 to 15 are formed in this manner. By forming the distal surface 31 of the proximal ridge segment 24P and the proximal surface 41 of the distal ridge segment 24D as described above, when the balloon 10 is bent, the balloon 10 can be bent more greatly if the specific notch 22X is located on the side surface of a bent portion of the balloon 10. It is preferable that the separation distance in the longitudinal axis direction between the distal surface 31 of the proximal ridge segment 24P and the proximal surface 41 of the distal ridge segment 24D be formed in this manner in any vertical cross section in the radial direction.

On the line of intersection with the virtual plane H, the distal surface 31 of the proximal ridge segment 24P may extend to be inclined proximally outward in the radial direction as illustrated in FIGS. 8 to 12, may extend in the radial direction as illustrated in FIG. 14, or may extend to be inclined distally outward in the radial direction as illustrated in FIG. 15. As illustrated in FIG. 13, on the line of intersection with the virtual plane H, the distal surface 31 of the proximal ridge segment 24P may have a portion extending in the radial direction and a portion extending to be inclined proximally outward in the radial direction. Although not illustrated in the drawings, on the line of intersection with the virtual plane H, the distal surface 31 of the proximal ridge segment 24P may have a portion extending to be inclined proximally outward in the radial direction and a portion extending to be inclined distally outward in the radial direction, or may have a portion extending to be inclined distally outward in the radial direction and a portion extending in the radial direction, or may have a portion extending to be inclined proximally outward in the radial direction, a portion extending to be inclined distally outward in the radial direction, and a portion extending in the radial direction.

On the line of intersection with the virtual plane H, the proximal surface 41 of the distal ridge segment 24D may extend to be inclined distally outward in the radial direction as illustrated in FIGS. 8 to 12, may extend in the radial direction as illustrated in FIG. 14, or may extend to be inclined proximally outward in the radial direction as illustrated in FIG. 15. As illustrated in FIG. 13, on the line of intersection with the virtual plane H, the proximal surface 41 of the distal ridge segment 24D may have a portion extending in the radial direction and a portion extending to be inclined distally outward in the radial direction. Although not illustrated in the drawings, on the line of intersection with the virtual plane H, the proximal surface 41 of the distal ridge segment 24D may have a portion extending to be inclined distally outward in the radial direction and a portion extending to be inclined proximally outward in the radial direction, or may have a portion extending to be inclined proximally outward in the radial direction and a portion extending in the radial direction, or may have a portion extending to be inclined distally outward in the radial direction, a portion extending to be inclined proximally outward in the radial direction, and a portion extending in the radial direction.

On the line of intersection with the virtual plane H, it is preferable that the distal surface 31 of the proximal ridge segment 24P extend in the radial direction or extend to be inclined proximally outward in the radial direction. It is more preferable that the distal surface 31 extend to be inclined proximally outward in the radial direction on at least a portion of the line of intersection with the virtual plane H. On the line of intersection with the virtual plane H, it is preferable that the proximal surface 41 of the distal ridge segment 24D extend in the radial direction or extend to be inclined distally outward in the radial direction. It is more preferable that the proximal surface 41 extend to be inclined distally outward in the radial direction on at least a portion of the line of intersection with the virtual plane H. By forming the end portions of the ridge segments 24 in this manner, when the balloon 10 is bent with the specific notch 22X on the inner side, the balloon 10 can be easily bent greatly.

The distal surface 31 of the proximal ridge segment 24P may extend to be inclined proximally outward in the radial direction on the line of intersection with the virtual plane H, and the proximal surface 41 of the distal ridge segment 24D may extend to be inclined distally outward in the radial direction on the line of intersection with the virtual plane H. FIGS. 26 to 29 illustrate configuration examples of cross-sectional views of the distal portion of the proximal ridge segment 24P and the proximal portion of the distal ridge segment 24D illustrated in FIGS. 8 to 12 taken along the line of intersection with the virtual plane H, and the distal surface 31 of the proximal ridge segment 24P and the proximal surface 41 of the distal ridge segment 24D are formed in this manner. In the case where the specific notch 22X is formed so as to extend from the top portion 21A to the base portion 21B of the ridge 21, the sectional views illustrated in FIG. 26 or FIG. 28 are referred to. In the case where the specific notch 22X is formed so as to extend from the top portion 21A of the ridge 21 to an intermediate position between the top portion 21A and the base portion 21B of the ridge 21, the sectional views illustrated in FIG. 27 or FIG. 29 are referred to. By forming the end portions of the ridge segments 24 in this manner, when the balloon 10 is bent with the specific notch 22X on the inner side, the balloon 10 can be easily bent greatly.

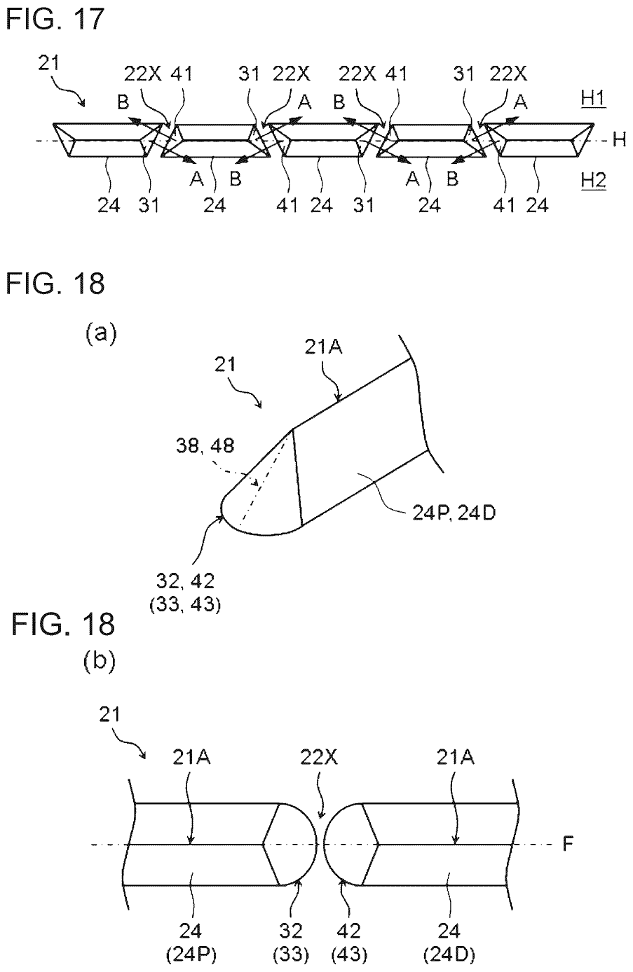

In the case where each of the ridges 21 is provided with the plurality of specific notches 22X, as viewed from the top portion 21A of the ridge 21, the extension directions of the plurality of specific notches 22X, which are arranged in the longitudinal axis direction on the ridge 21, that is, the extension directions of the distal surfaces 31 of the proximal ridge segments 24P and the extension directions of the proximal surfaces 41 of the distal ridge segments 24D, may be the same as each other or may be different from each other. FIGS. 16 and 17 are plan views of one of the ridges 21 as viewed from the top portion 21A side and illustrate configuration examples of the ridge 21 in which the plurality of specific notches 22X are arranged in the longitudinal axis direction. For example, as illustrated in FIG. 16, when one side of the virtual plane H in the ridge 21 is defined as a first side H1 and the other side of the virtual plane H is defined as a second side H2, in all of the specific notches 22X provided in the ridge 21, the normal vector A may be oriented toward the distal side on the first side H1, and the normal vector B may be oriented toward the proximal side on the second side H2. Alternatively, as illustrated in FIG. 17, the specific notches 22X having the normal vector A oriented toward the distal side on the first side H1 and the normal vector B oriented toward the proximal side on the second side H2 and the specific notches 22X having the normal vector A oriented toward the distal side on the second side H2 and the normal vector B oriented toward the proximal side on the first side H1 may be provided in the ridges 21 so as to be alternately arranged in the longitudinal axis direction. In this manner, the extension directions of the specific notches 22X can be set in various ways.

The configuration of one of the ridges 21 provided on the balloon 10 of the second embodiment, in particular, the configuration of end portions of the ridge segments 24 that are adjacent to the specific notch 22X satisfying Requirement B, will be described with reference to FIGS. 18 to 25. FIGS. 18 to 25 illustrate various configuration examples of the end portions of the ridge segments 24 adjacent to the specific notch 22X satisfying Requirement B. Each of FIGS. 18(a) to 25(a) is a perspective view of a distal end portion of the proximal ridge segment 24P as viewed from the distal side or a perspective view of a proximal end portion of the distal ridge segment 24D as viewed from the proximal side. Each of FIGS. 18(b) to 25(b) is a plan view of the distal end portion of the proximal ridge segment 24P and the proximal end portion of the distal ridge segment 24D as viewed from the top portion 21A of the corresponding ridge 21.

In each of the ridges 21 of the balloon 10 according to the second embodiment, the distal end portion of the proximal ridge segment 24P and the proximal end portion of the distal ridge segment 24D adjacent to the specific notch 22X are formed as follows. In a plan view as viewed from the top portion 21A of the ridge 21, a distal end edge 32 of the proximal ridge segment 24P adjacent to the specific notch 22X on the proximal side has a protruding shape protruding toward the distal side, and a proximal end edge 42 of the distal ridge segment 24D adjacent to the specific notch 22X on the distal side has a protruding shape protruding toward the proximal side. Even when the specific notch 22X is formed as described above, the flexibility of the balloon 10 can be enhanced. To be specific, in the case where the balloon 10 is bent with the specific notch 22X on the inner side, when the distal end portion of the proximal ridge segment 24P and the proximal end portion of the distal ridge segment 24D adjacent to the specific notch 22X come into contact with each other, the tips of the protruding shapes are more easily displaced from each other. Therefore, the balloon 10 can be easily bent greatly. Alternatively, when the balloon 10 is bent, in the case where the specific notch 22X is positioned on the side surface of a bent portion of the balloon 10 instead of the inner side of the bent balloon 10, the balloon 10 can be bent more greatly.

The distal end edge 32 of the proximal ridge segment 24P refers to the outer edge of a portion of the proximal ridge segment 24P that faces the distal side in a plan view of the ridge 21 as viewed from the top portion 21A side. The proximal end edge 42 of the distal ridge segment 24D refers to the outer edge of a portion of the distal ridge segment 24D that faces the proximal side in a plan view of the ridge 21 as viewed from the top portion 21A side. The plan view of the ridge 21 as viewed from the top portion 21A side refers to a plan view of the ridge 21 as viewed from the radially outer side.

The distal end edge 32 of the proximal ridge segment 24P may be composed of a straight line, a curved line, or a combination thereof, as long as it has a protruding shape protruding toward the distal side. The proximal end edge 42 of the distal ridge segment 24D may be composed of a straight line, a curved line, or a combination thereof, as long as it has a protruding shape protruding toward the proximal side. When the distal end edge 32 of the proximal ridge segment 24P and the proximal end edge 42 of the distal ridge segment 24D are composed of straight lines, each of these end edges is composed of two or more straight lines. The protruding shape of the distal end edge 32 of the proximal ridge segment 24P may be the same as or different from the protruding shape of the proximal end edge 42 of the distal ridge segment 24D. In addition, the protruding shapes of the distal end edges 32 of the plurality of proximal ridge segments 24P may be the same as or different from each other, and the protruding shapes of the proximal end edges 42 of the plurality of distal ridge segments 24D may be the same as or different from each other.

As illustrated in FIGS. 18, 19, and 21, the protruding shape of the distal end edge 32 of the proximal ridge segment 24P may have a curved portion 33 curved toward the distal side, and the protruding shape of the proximal end edge 42 of the distal ridge segment 24D may have a curved portion 43 curved toward the proximal side. By forming the end portions of the ridge segments 24 in this manner, the inner wall of a blood vessel is less likely to be damaged even if the end portions of the ridge segments 24 come into contact with the inner wall of the blood vessel when the balloon 10 is bent. Accordingly, the balloon 10 having favorable safety can be provided. In the case where the protruding shape of the distal end edge 32 of the proximal ridge segment 24P has the curved portion 33, the curved portion 33 curved toward the distal side may be positioned on the distal side with respect to a line segment connecting both ends of the curved portion 33. When the protruding shape of the proximal end edge 42 of the distal ridge segment 24D has the curved portion 43, the curved portion 43 curved toward the proximal side may be positioned on the proximal side with respect to a line segment connecting both ends of the curved portion 43.

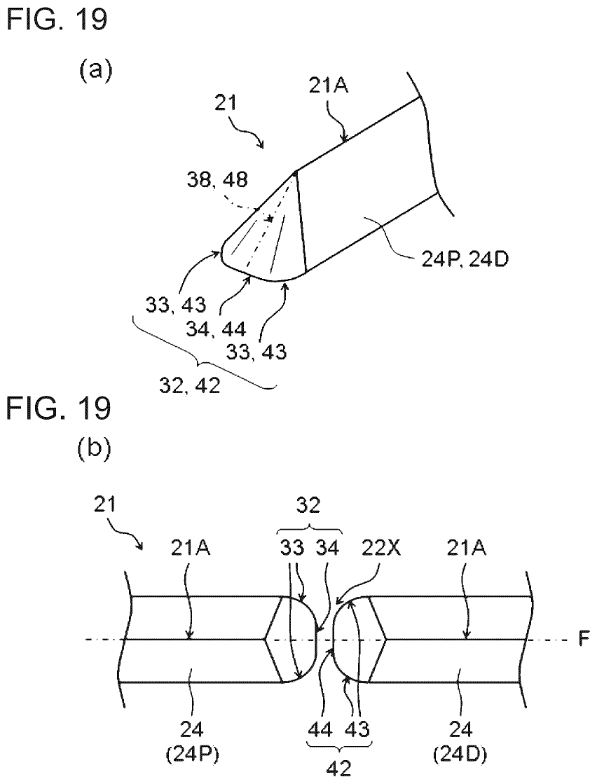

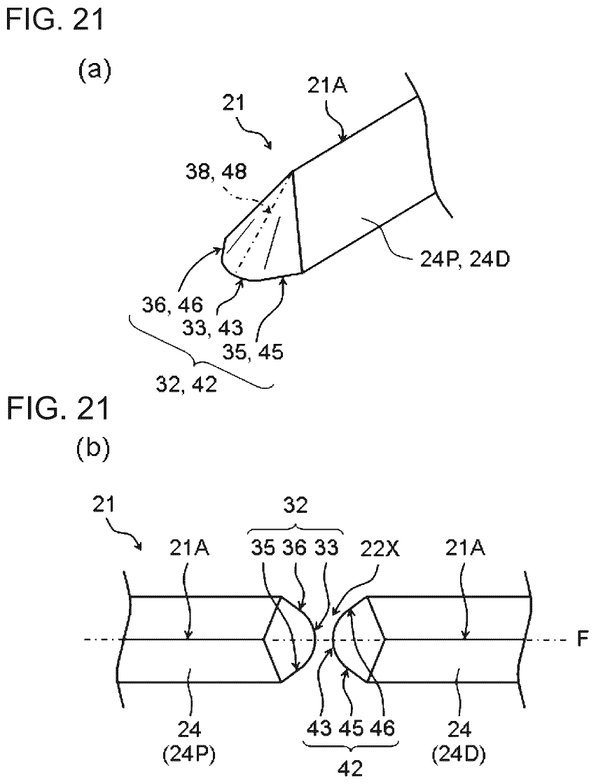

When the protruding shape of the distal end edge 32 of the proximal ridge segment 24P and/or the protruding shape of the proximal end edge 42 of the distal ridge segment 24D has a curved portion, the entire protruding shape may be composed of the curved portion, or only a portion of the protruding shape may be composed of the curved portion. In the latter case, the protruding shape may be composed of the curved portion and a straight portion, and the distal end edge 32 of the proximal ridge segment 24P and/or the proximal end edge 42 of the distal ridge segment 24D may be formed smoothly from the curved portion to the straight portion (i.e., it is preferable that, in a plan view of the ridge 21 as viewed from the top portion 21A side, the inclination at the connecting portion between the curved portion and the straight portion continuously change). In FIG. 18, the protruding shape of the distal end edge 32 of the proximal ridge segment 24P is composed only of the curved portion 33, and the protruding shape of the proximal end edge 42 of the distal ridge segment 24D is composed only of the curved portion 43. In FIG. 19, the protruding shape of the distal end edge 32 of the proximal ridge segment 24P is composed of the curved portion 33 and a straight portion 34. The protruding shape of the proximal end edge 42 of the distal ridge segment 24D is composed of the curved portion 43 and a straight portion 44. In FIG. 21, the protruding shape of the distal end edge 32 of the proximal ridge segment 24P is composed of the curved portion 33, a proximal first straight portion 35, and a proximal second straight portion 36. The protruding shape of the proximal end edge 42 of the distal ridge segment 24D is composed of the curved portion 43, a distal first straight portion 45, and a distal second straight portion 46.

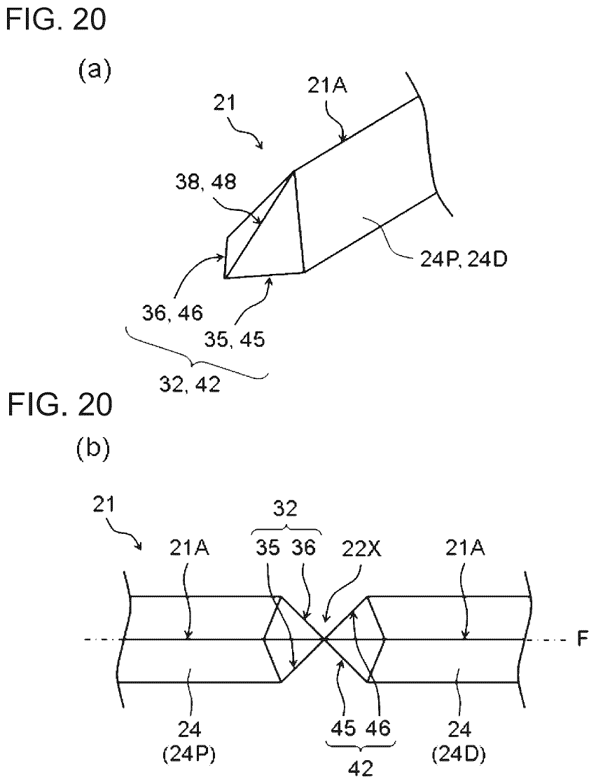

As illustrated in FIGS. 20 to 22, as viewed from the top portion 21A of the ridge 21, the protruding shape of the distal end edge 32 of the proximal ridge segment 24P may have a proximal first straight portion 35 located on one side of a virtual straight line F and a proximal second straight portion 36 located on the other side of the virtual straight line F, the virtual straight line F passing through the top portion 21A of the ridge 21 and extending in the longitudinal axis direction. The proximal first straight portion 35 and the proximal second straight portion 36 may be configured to extend in such a manner as to move away from the virtual straight line F with increasing distance toward the proximal side. Furthermore, as viewed from the top portion 21A of the ridge 21, the protruding shape of the proximal end edge 42 of the distal ridge segment 24D may include a distal first straight portion 45 located on one side of the virtual straight line F and a distal second straight portion 46 located on the other side of the virtual straight line F. The distal first straight portion 45 and the distal second straight portion 46 may be configured to extend in such a manner as to move away from the virtual straight line F with increasing distance toward the distal side. By forming the end portions of the ridge segments 24 in this manner, when the balloon 10 is bent, the balloon 10 can be bent more greatly if the specific notch 22X is located on the side surface of a bent portion of the balloon 10.

When the distal end edge 32 of the proximal ridge segment 24P and/or the proximal end edge 42 of the distal ridge segment 24D are formed as described above, the proximal first straight portion 35 may be directly connected to the proximal second straight portion 36. The proximal first straight portion 35 and the proximal second straight portion 36 may be connected to each other via another straight portion or a curved portion. The distal first straight portion 45 may be directly connected to the distal second straight portion 46. The distal first straight portion 45 and the distal second straight portion 46 may be connected to each other via another straight portion or a curved portion. FIGS. 20 to 22 illustrate configuration examples of the distal end edge 32 of the proximal ridge segment 24P and the proximal end edge 42 of the distal ridge segment 24D formed in this manner, and each of them will be described below.

In FIG. 20, the protruding shape of the distal end edge 32 of the proximal ridge segment 24P is formed by connecting a distal end of the proximal first straight portion 35 to a distal end of the proximal second straight portion 36, and the protruding shape of the proximal end edge 42 of the distal ridge segment 24D is formed by connecting a proximal end of the distal first straight portion 45 to a proximal end of the distal second straight portion 46. By forming the end portions of the ridge segments 24 in this manner, when the balloon 10 is bent with the specific notch 22X on the inner side, the distal portion of the proximal ridge segment 24P and the proximal portion of the distal ridge segment 24D are more easily displaced from each other and more likely to come into contact with each other, and the balloon 10 can be easily bent greatly.

In FIG. 21, the protruding shape of the distal end edge 32 of the proximal ridge segment 24P has the curved portion 33 between the proximal first straight portion 35 and the proximal second straight portion 36, and the protruding shape of the proximal end edge 42 of the distal ridge segment 24D has the curved portion 43 between the distal first straight portion 45 and the distal second straight portion 46. The curved portion 33 included in the protruding shape of the distal end edge 32 of the proximal ridge segment 24P is curved toward the distal side, and the curved portion 43 included in the protruding shape of the proximal end edge 42 of the distal ridge segment 24D is curved toward the proximal side. By forming the end portions of the ridge segments 24 in this manner, the inner wall of a blood vessel is less likely to be damaged even if the end portions of the ridge segments 24 come into contact with the inner wall of the blood vessel when the balloon 10 is bent.

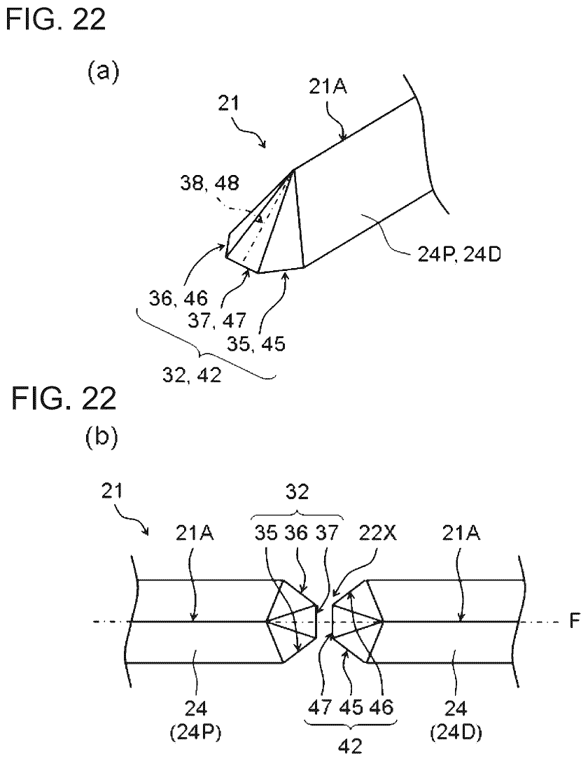

In FIG. 22, the protruding shape of the distal end edge 32 of the proximal ridge segment 24P has a proximal third straight portion 37 between the proximal first straight portion 35 and the proximal second straight portion 36, and the proximal first straight portion 35 and the proximal second straight portion 36 are located on the proximal side of the proximal third straight portion 37. The protruding shape of the proximal end edge 42 of the distal ridge segment 24D has a distal third straight portion 47 between the distal first straight portion 45 and the distal second straight portion 46, and the distal first straight portion 45 and the distal second straight portion 46 are located on the distal side of the distal third straight portion 47. Even if the end portions of the ridge segments 24 are formed in this manner, the inner wall of a blood vessel is less likely to be damaged even in the case where the end portions of the ridge segments 24 come into contact with the inner wall of the blood vessel when the balloon 10 is bent. In this case, it is only necessary that at least a portion of the proximal first straight portion 35 and at least a portion of the proximal second straight portion 36 be located on the proximal side with respect to the proximal third straight portion 37, and it is only necessary that at least a portion of the distal first straight portion 45 and at least a portion of the distal second straight portion 46 be located on the distal side with respect to the distal third straight portion 47.

In a plan view as viewed from the top portion 21A of the ridge 21, it is preferable that the proximal third straight portion 37 extend such that an angle formed between the proximal third straight portion 37 and the longitudinal axis direction is larger than an angle formed between the proximal first straight portion 35 and the longitudinal axis direction and than an angle formed between the proximal second straight portion 36 and the longitudinal axis direction. In a plan view as viewed from the top portion 21A of the ridge 21, it is preferable that the distal third straight portion 47 extend such that an angle formed between the distal third straight portion 47 and the longitudinal axis direction is larger than an angle formed between the distal first straight portion 45 and the longitudinal axis direction and than an angle formed between the distal second straight portion 46 and the longitudinal axis direction. Each of the angles formed between the proximal first straight portion 35 and the longitudinal axis direction, between the proximal second straight portion 36 and the longitudinal axis direction, between the distal first straight portion 45 and the longitudinal axis direction, and between the distal second straight portion 46 and the longitudinal axis direction is in the range of more than 0° and less than 90°, and each of the angles formed between the proximal third straight portion 37 and the longitudinal axis direction and between the distal third straight portion 47 and the longitudinal axis direction is in the range of more than 0° and 90° or less. When each of the angles formed between the proximal third straight portion 37 and the longitudinal axis direction and between the distal third straight portion 47 and the longitudinal axis direction is 90°, the proximal third straight portion 37 and the distal third straight portion 47 extend perpendicularly to the longitudinal axis direction. Each of the angles formed between the proximal third straight portion 37 and the longitudinal axis direction and between the distal third straight portion 47 and the longitudinal axis direction may be 60° or more and 90° or less, or 75° or more and 90° or less. Thus, the inner wall of a blood vessel is less likely to be damaged when the end portions of the ridge segments 24 come into contact with the inner wall of the blood vessel. It is more preferable that the proximal third straight portion 37 and/or the distal third straight portion 47 extend perpendicularly to the longitudinal axis direction, and it is particularly preferable that both the proximal third straight portion 37 and the distal third straight portion 47 extend perpendicularly to the longitudinal axis direction. Each of the angles formed between the proximal first straight portion 35 and the longitudinal axis direction, between the proximal second straight portion 36 and the longitudinal axis direction, between the distal first straight portion 45 and the longitudinal axis direction, and between the distal second straight portion 46 and the longitudinal axis direction may be 20° or more, or 30° or more, and may be 70° or less, or 60° or less.

In a plan view as viewed from the top portion 21A of the ridge 21, the most distal portion of the distal end edge 32 of the proximal ridge segment 24P may be on the virtual straight line F passing through the top portion 21A of the ridge 21 and extending in the longitudinal axis direction. In a plan view as viewed from the top portion 21A of the ridge 21, the most proximal portion of the proximal end edge 42 of the distal ridge segment 24D may be on the virtual straight line F passing through the top portion 21A of the ridge 21 and extending in the longitudinal axis direction. In FIGS. 18 to 22, 24, and 25, the distal end edge 32 of the proximal ridge segment 24P and the proximal end edge 42 of the distal ridge segment 24D are formed in this manner. By forming the end portions of the ridge segments 24 in this manner, when the balloon 10 is bent, the balloon 10 can be easily bent greatly regardless of the bending direction of the balloon 10 and of whether the specific notch 22X is located on the inner side or on the side surface of the bent portion of the balloon 10.

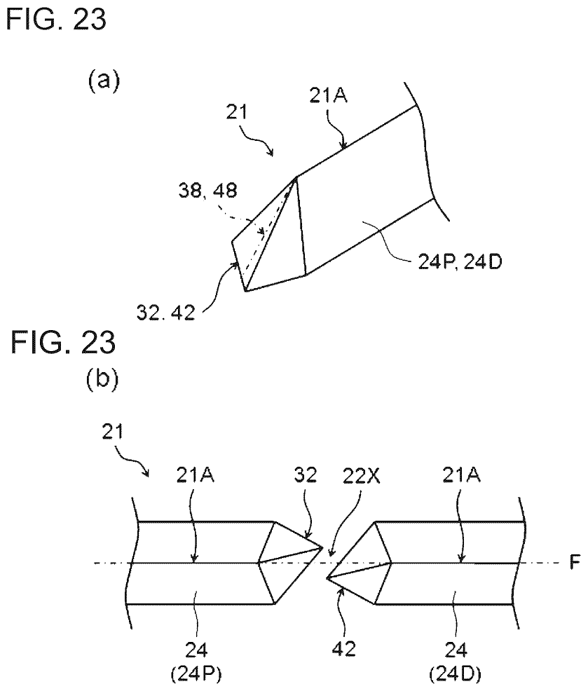

In contrast, the protruding shape of the distal end edge 32 of the proximal ridge segment 24P and the protruding shape of the proximal end edge 42 of the distal ridge segment 24D may be configured as illustrated in FIG. 23. That is, in a plan view as viewed from the top portion 21A of the ridge 21, the most distal portion of the distal end edge 32 of the proximal ridge segment 24P may be located on one side of the virtual straight line F passing through the top portion 21A of the ridge 21 and extending in the longitudinal axis direction, and the most proximal portion of the proximal end edge 42 of the distal ridge segment 24D may be located on the other side with respect to the virtual straight line F passing through the top portion 21A of the ridge 21 and extending in the longitudinal axis direction. In this case, the most distal portion of the distal end edge 32 of the proximal ridge segment 24P and the most proximal portion of the proximal end edge 42 of the distal ridge segment 24D are positioned so as to be displaced from each other in the width direction of the ridge 21. This makes it easier to greatly bend the balloon 10 especially when the balloon 10 is bent with the specific notch 22X on the inner side. In FIG. 23, the specific notch 22X is configured so as to satisfy Requirement A.

The outer edge shape of the distal end portion of the proximal ridge segment 24P at an arbitrary position in the radial direction (i.e., the outer edge shape of the distal end portion of the proximal ridge segment 24P obtained when the ridge 21 is cut by an arbitrary plane parallel to the outer surface of the balloon main body 16) may be similar or dissimilar in shape to the distal end edge 32. For example, the distal end edge 32 of the proximal ridge segment 24P may be composed of a curved portion, and the outer edge shape of the distal end portion of the proximal ridge segment 24P at an arbitrary position in the radial direction may be composed of a straight portion. Similarly, the outer edge shape of the proximal end portion of the distal ridge segment 24D at an arbitrary position in the radial direction may be similar or dissimilar in shape to the proximal end edge 42.

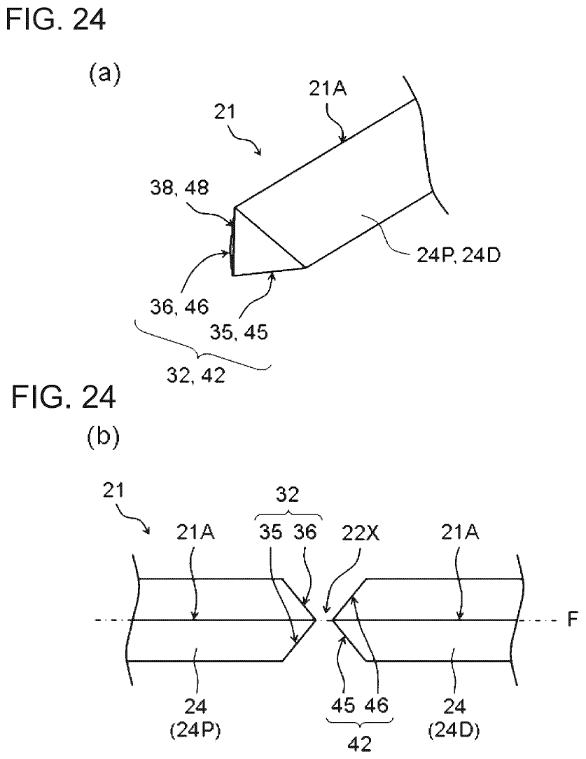

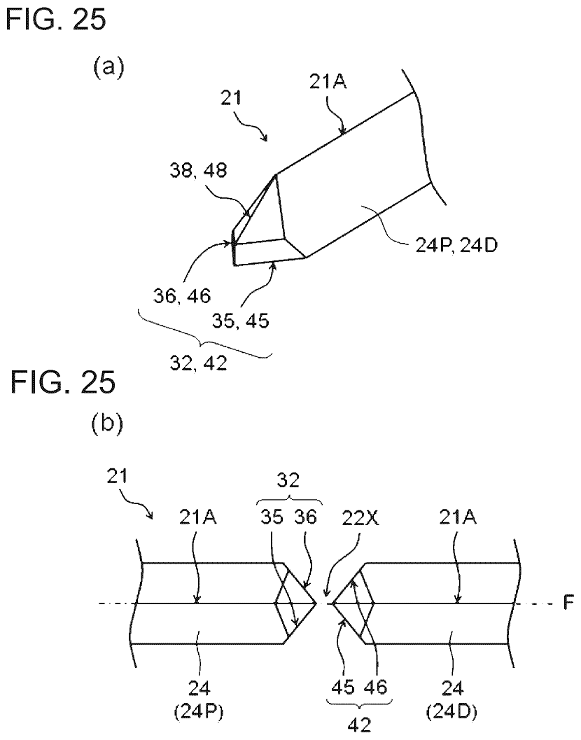

In a cross section along the longitudinal axis direction and passing through the top portion 21A of the ridge 21 (specifically, a cross-sectional view of the ridge 21 taken by a virtual plane passing through the top portion 21A of the ridge 21 and formed by the longitudinal axis direction and the radial direction), the distal outer edge 38 of the proximal ridge segment 24P may extend to be inclined proximally outward in the radial direction as illustrated in FIGS. 18 to 23, or may extend in the radial direction as illustrated in FIG. 24. Furthermore, as illustrated in FIG. 25, the distal outer edge 38 of the proximal ridge segment 24P may have a portion extending in the radial direction and a portion extending to be inclined proximally outward in the radial direction. Although not illustrated in the drawings, the distal outer edge 38 of the proximal ridge segment 24P may have a portion extending to be inclined distally outward in the radial direction.

In a cross section along the longitudinal axis direction and passing through the top portion 21A of the ridge 21, the proximal outer edge 48 of the distal ridge segment 24D may extend to be inclined distally outward in the radial direction as illustrated in FIGS. 18 to 23, or may extend in the radial direction as illustrated in FIG. 24. Furthermore, as illustrated in FIG. 25, the proximal outer edge 48 of the distal ridge segment 24D may have a portion extending in the radial direction and a portion extending to be inclined distally outward in the radial direction. Although not illustrated in the drawings, the proximal outer edge 48 of the distal ridge segment 24D may have a portion extending to be inclined proximally outward in the radial direction.

In a cross section along the longitudinal axis direction and passing through the top portion 21A of the ridge 21, it is preferable that the distal outer edge 38 of the proximal ridge segment 24P extend to be inclined proximally outward in the radial direction, and that the proximal outer edge 48 of the distal ridge segment 24D extend to be inclined distally outward in the radial direction. FIGS. 26 to 29 illustrate configuration examples of cross-sectional views, along the longitudinal axis direction and passing through the top portion 21A of the ridge 21, for the distal end portion of the proximal ridge segment 24P and for the proximal end portion of the distal ridge segment 24D, which are illustrated in FIGS. 18 to 23. The distal outer edge 38 of the proximal ridge segment 24P and the proximal outer edge 48 of the distal ridge segment 24D are formed in this manner. In the case where the specific notch 22X is formed so as to extend from the top portion 21A to the base portion 21B of the ridge 21, the sectional view illustrated in FIG. 26 or FIG. 28 is referred to. In the case where the specific notch 22X is formed so as to extend from the top portion 21A of the ridge 21 to an intermediate position between the top portion 21A and the base portion 21B of the ridge 21, the sectional view illustrated in FIG. 27 or FIG. 29 is referred to. By forming the end portions of the ridge segments 24 in this manner, when the balloon 10 is bent with the specific notch 22X on the inner side, the balloon 10 can be easily bent greatly. In this case, the shape of the distal end edge 32 of the proximal ridge segment 24P and the shape of the proximal end edge 42 of the distal ridge segment 24D as viewed from the top portion 21A of the ridge 21 are defined by the bottom portion 22B of the specific notch 22X.

In the straight tube portion 13 of the balloon 10 according to the first embodiment and in the straight tube portion 13 of the balloon 10 according to the second embodiment, only some of the notches 22 provided in the ridges 21 may be formed as the specific notches 22X, or all of the notches 22 may be formed as the specific notches 22X. It is preferable that a larger number of the notches 22 provided in the ridges 21 be formed as the specific notches 22X. For example, it is preferable that ¼ or more of the notches 22 provided in the ridges 21 be formed as the specific notches 22X. It is more preferable that ⅓ or more of the notches 22 be formed as the specific notches 22X. It is further preferable that ½ or more of the notches 22 be formed as the specific notches 22X. It is still further preferable that ⅔ or more of the notches 22 be formed as the specific notches 22X.

At least one of the specific notches 22X may be provided in each of the ridges 21. This enables the balloon 10 to be easily bent in any radial direction.

The specific notches 22X may be provided at least in the distal sections 19 of the ridges 21. By providing the specific notches 22X in the distal sections 19 of the ridges 21, it becomes easier to push the balloon 10 forward to be inserted into a curved portion of a blood vessel. In this case, it is only necessary that at least one of the specific notches 22X be provided in the distal section 19 of at least one of the ridges 21, however, it is preferable that the specific notches 22X be provided in the distal sections 19 of all the ridges 21. Furthermore, all of the notches 22 provided in the distal sections 19 of the ridges 21 may be formed as the specific notches 22X.

It is also preferable that the specific notches 22X be provided in the proximal sections 17 of the ridges 21. By providing the specific notches 22X in the proximal sections 17 of the ridges 21, it becomes easier to withdraw the balloon 10 and pass it through a curved portion. In this case, it is only necessary that at least one of the specific notches 22X be provided in the proximal section 17 of at least one of the ridges 21, however, it is preferable that the specific notches 22X be provided in the proximal sections 17 of all the ridges 21. Also, all of the notches 22 provided in the proximal sections 17 of the ridges 21 may be formed as the specific notches 22X.

The specific notches 22X may be provided in both the distal sections 19 and the proximal sections 17 of the ridges 21, and may also be provided in the intermediate sections 18 of the ridges 21. By providing the specific notches 22X also in the intermediate sections 18 of the ridges 21, the flexibility of the balloon 10 can be further enhanced.

In the balloon 10 according to the first embodiment and in the balloon 10 according to the second embodiment, when at least one of the ridges 21 is further provided on at least one of the proximal sleeve portion 11, the proximal tapered portion 12, the distal tapered portion 14, and the distal sleeve portion 15, at least one of the specific notches 22X may also be provided in the at least one ridge 21 provided on the proximal sleeve portion 11, the proximal tapered portion 12, the distal tapered portion 14, or the distal sleeve portion 15. This makes it easier to greatly bend the balloon 10 at a portion thereof other than the straight tube portion 13.

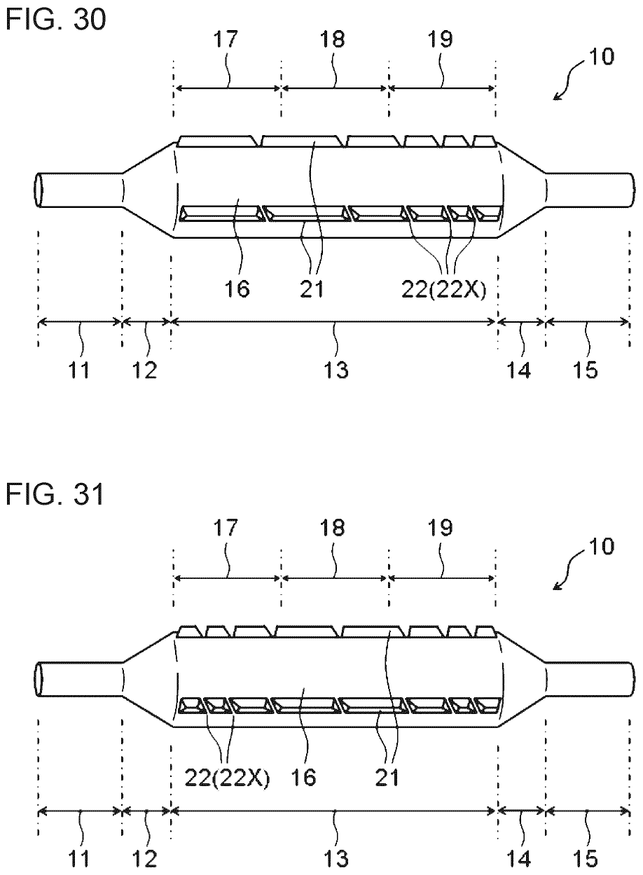

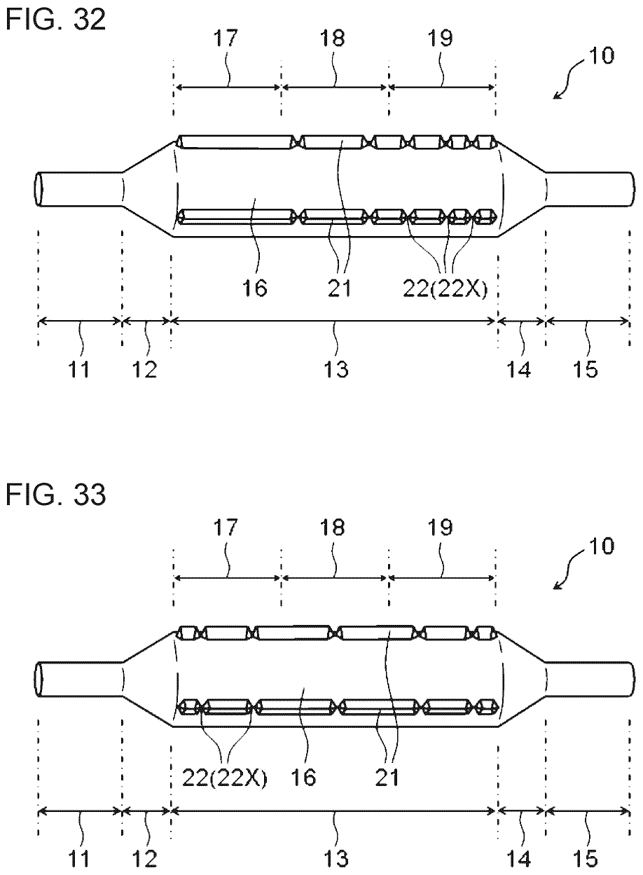

In the configuration example of the balloon 10 illustrated in FIGS. 4 and 5, the notches 22 are provided, at substantially equal intervals, in the ridges 21, but the notches 22 may be provided, at unequal intervals in the longitudinal axis direction, in the ridges 21. FIGS. 30 to 33 illustrate other configuration examples of the ridges 21 provided on the balloon 10. FIG. 30 and FIG. 31 illustrate a modification of the balloon 10 illustrated in FIG. 4, that is, a configuration example of the balloon 10 provided with the ridges 21 having the specific notches 22X satisfying Requirement A. FIG. 32 and FIG. 33 illustrate a modification of the balloon 10 illustrated in FIG. 5, that is, a configuration example of the balloon 10 provided with the ridges 21 having the specific notches 22X satisfying Requirement B.

As illustrated in FIGS. 30 and 32, the notches 22 may be provided in each of the ridges 21 such that the number of the notches 22 provided in the distal section 19 is greater than the number of the notches 22 provided in the intermediate section 18. As a result, the flexibility of the distal portion of the balloon 10 can be increased, and it becomes easier to push the balloon 10 forward to be inserted into a curved portion. In addition, by providing a smaller number of the notches 22 or providing no notches 22 in the intermediate sections 18 of the ridges 21, the scoring function in the intermediate sections 18 of the ridges 21 can be enhanced.

As illustrated in FIGS. 31 and 33, each of the ridges 21 may be provided with the notches 22 such that the number of the notches 22 provided in the distal section 19 is greater than the number of the notches 22 provided in the intermediate section 18 and such that the number of the notches 22 provided in the proximal section 17 is greater than the number of the notches 22 provided in the intermediate section 18. As a result, both when the balloon 10 is pushed forward to be inserted into a curved portion and when the balloon 10 is withdrawn to pass through the curved portion, it becomes easier to maneuver the balloon 10 through the curved portion. In addition, the scoring function in the intermediate sections 18 of the ridges 21 can be enhanced.

When the ridges 21 are provided with the plurality of notches 22, the depths of the notches 22 may be the same as or different from each other. In one embodiment, the notches 22 may be provided in the ridges 21 as follows. That is, in the ridges 21, the notches 22 may be formed such that the deepest depth of the notches 22 provided in the distal sections 19 is deeper than the deepest depth of the notches 22 provided in the intermediate sections 18. As a result, the flexibility of the distal portion of the balloon 10 can be increased, and it becomes easier to push the balloon 10 forward to be inserted into a curved portion.

The notches 22 may be formed in the ridges 21 such that the deepest depth of the notches 22 provided in the distal sections 19 is deeper than the deepest depth of the notches 22 provided in the intermediate sections 18 and such that the deepest depth of the notches 22 provided in the proximal sections 17 is deeper than the deepest depth of the notches 22 provided in the intermediate sections 18. In this case, the flexibility of the distal and proximal portions of the balloon 10 can be increased, and both when the balloon 10 is pushed forward to be inserted into a curved portion and when the balloon 10 is withdrawn to pass through the curved portion, it becomes easier to maneuver the balloon 10 through the curved portion.

Contrary to the above, the notches 22 may be provided in the ridges 21 as follows. That is, in the ridges 21, the notches 22 may be formed such that the deepest depth of the notches 22 provided in the intermediate sections 18 is deeper than the deepest depth of the notches 22 provided in the distal sections 19. Alternatively, in the ridges 21, the notches 22 may be formed such that the deepest depth of the notches 22 provided in the intermediate sections 18 is deeper than the deepest depth of the notches 22 provided in the distal sections 19 and deeper than the deepest depth of the notches 22 provided in the proximal sections 17. By providing the notches 22 in the ridges 21 as described above, it is possible to increase the flexibility in the intermediate sections 18 while reducing the number of the notches 22 provided in the intermediate sections 18. Therefore, it becomes easier to ensure the flexibility of the entire balloon 10 in the longitudinal axis direction while ensuring the scoring function provided by the ridges 21.

The length of the straight tube portion 13 of the balloon 10 in the longitudinal axis direction may be, for example, 4 mm or more, 10 mm or more, 20 mm or more, or 30 mm or more. However, as the length in the longitudinal axis direction increases, the balloon 10 provided with the ridges 21 becomes more difficult to pass through a curved portion. Therefore, from the viewpoint of more effectively achieving the effect of providing the notches 22 in the ridges 21, the length of the straight tube portion 13 of the balloon 10 in the longitudinal axis direction may be 50 mm or more, 60 mm or more, or 80 mm or more.

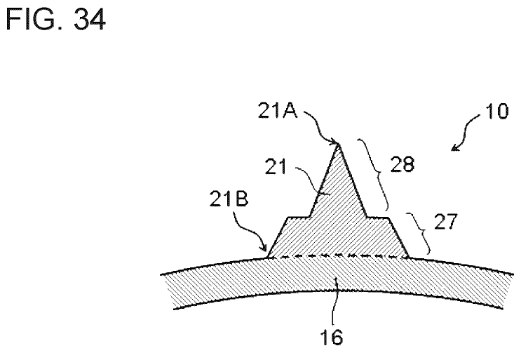

As illustrated in FIG. 34, each of the ridges 21 may be formed so as to narrow in a stepwise manner toward the top portion 21A in a vertical cross section in the longitudinal axis direction of the straight tube portion 13, and the notches 22 may be formed only in portions on the top portions 21A side of the ridges 21, which are formed in a step-like shape. For example, each of the ridges 21 may include a first stepped portion 27 adjacent to the outer surface of the balloon main body 16 and a second stepped portion 28 that is closer to the top portion 21A than the first stepped portion 27 is. The notches 22 may be formed in the second stepped portions 28 of the ridges 21 but not be formed in the first stepped portions 27 of the ridges 21. Alternatively, in each of the ridges 21, the notch 22 that is deeper may be formed so as to extend across the second stepped portion 28 and the first stepped portion 27, and the notch 22 that is shallower may be formed in the second stepped portion 28 but not in the first stepped portion 27. The first stepped portion 27 and the second stepped portion 28 may be made of the same material or may be made of different materials from each other. For example, both the first stepped portion 27 and the second stepped portion 28 may be made of a resin, or the first stepped portion 27 may be made of a resin while the second stepped portion 28 may be made of a metal.

A drug may be held on the outer surface of the straight tube portion 13 of the balloon 10. The drug is not particularly limited as long as it is a pharmacologically active substance, and examples of such drugs include pharmaceutically acceptable drugs, such as gene therapy drugs, non-gene therapy drugs, small molecules, and cells. In particular, when a catheter is used for the purpose of suppressing restenosis of a blood vessel after treatment in angioplasty, an anti-restenosis agent, such as an antiproliferative agent or an immunosuppressive agent, may be used as the drug. Examples of such drugs include paclitaxel, sirolimus (rapamycin), everolimus, and zotarolimus.

This application claims the benefit of priority based on Japanese Patent Application Nos. 2023-109026 and 2023-109027, filed on Jul. 3, 2023. The entire contents of the specifications of Japanese Patent Application Nos. 2023-109026 and 2023-109027, filed on Jul. 3, 2023, are incorporated herein by reference.

Although the disclosure has been described with respect to only a limited number of embodiments, those skilled in the art, having benefit of this disclosure, will appreciate that various other embodiments may be devised without departing from the scope of the present disclosure. Accordingly, the scope of the invention should be limited only by the attached claims.

### REFERENCE SIGNS LIST

1 balloon catheter
2 shaft
3 inner shaft
4 outer shaft, 4A proximal outer shaft, 4B distal outer shaft
5 hub
6 fluid injection portion
7 guidewire port
8 distal tip
9 radiopaque marker
10 balloon
11 proximal sleeve portion
12 proximal tapered portion
13 straight tube portion
14 distal tapered portion
15 distal sleeve portion
16 balloon main body
17 proximal section
18 intermediate section
19 distal section
21 ridge, 21A top portion, 21B base portion
22 notch, 22A top portion, 22B base portion, 22X specific notch
23 interrupted portion
24 ridge segment, 24P proximal ridge segment, 24D distal ridge segment
25 ridge-present region
26 ridge-absent region
27 first stepped portion
28 second stepped portion
31 distal surface (of proximal ridge segment)
32 distal end edge (of proximal ridge segment)
33 curved portion (of distal end edge of proximal ridge segment)
34 straight portion (of distal end edge of proximal ridge segment)
35 proximal first straight portion
36 proximal second straight portion
37 proximal third straight portion
38 distal outer edge (of proximal ridge segment in cross section along longitudinal axis direction)
41 proximal surface (of distal ridge segment)
42 proximal end edge (of distal ridge segment)
43 curved portion (of proximal end edge of distal ridge segment)
44 straight portion (of proximal end edge of distal ridge segment)
45 distal first straight portion
46 distal second straight portion
47 distal third straight portion
48 proximal outer edge (of distal ridge segment in cross section along longitudinal axis direction)
H virtual plane
H1 first side
H2 second side
F virtual straight line

1. A balloon for a balloon catheter having a longitudinal axis direction extending from a proximal side to a distal side and a radial direction orthogonal to the longitudinal axis direction,
wherein the balloon comprises a straight tube portion, a proximal tapered portion located on a proximal side of the straight tube portion, and a distal tapered portion located on a distal side of the straight tube portion,
wherein the straight tube portion includes a balloon main body having a cylindrical shape, and a ridge provided on an outer surface of the balloon main body, the ridge protruding outward in the radial direction and extending in the longitudinal axis direction,
wherein at least one notch is formed in the ridge, and the ridge is divided into a plurality of ridge segments by the at least one notch, and
wherein the at least one notch includes at least one specific notch satisfying a requirement that
a distal surface of one of the ridge segments (hereinafter referred to as a “proximal ridge segment”) that is adjacent to a proximal side of the at least one specific notch has a normal vector A on a line of intersection with a virtual plane formed by the longitudinal axis direction and the radial direction and passing through a top portion of the ridge, the normal vector A being oriented toward a distal side on one side of the virtual plane, and
a proximal surface of another one of the ridge segments (hereinafter referred to as a “distal ridge segment”) that is adjacent to a distal side of the at least one specific notch has a normal vector B on a line of intersection with the virtual plane, the normal vector B being oriented toward a proximal side on another side of the virtual plane.

2. A balloon for a balloon catheter having a longitudinal axis direction extending from a proximal side to a distal side and a radial direction orthogonal to the longitudinal axis direction,
wherein the balloon comprises a straight tube portion, a proximal tapered portion located on a proximal side of the straight tube portion, and a distal tapered portion located on a distal side of the straight tube portion,
wherein the straight tube portion includes a balloon main body having a cylindrical shape, and a ridge provided on an outer surface of the balloon main body, the ridge protruding outward in the radial direction and extending in the longitudinal axis direction,
wherein a notch is formed in the ridge, and the ridge is divided into a plurality of ridge segments by the notch, and
wherein the notch includes a specific notch satisfying a requirement that, in a plan view from a top portion of the ridge, a distal end edge of one of the ridge segments (hereinafter referred to as a “proximal ridge segment”) that is adjacent to a proximal side of the specific notch has a protruding shape protruding toward a distal side, and a proximal end edge of another one of the ridge segments (hereinafter referred to as a “distal ridge segment”) that is adjacent to a distal side of the specific notch has a protruding shape protruding toward a proximal side.

3. The balloon according to claim 1,
wherein the ridge has a plurality of the specific notches arranged in the longitudinal axis direction, and
wherein, when one side of the virtual plane in the ridge is defined as a first side and another side of the virtual plane is defined as a second side, in all of the specific notches provided in the ridge, the normal vector A is oriented toward a distal side on the first side, and the normal vector B is oriented toward a proximal side on the second side.

4. The balloon according to claim 1,
wherein the ridge has a plurality of the specific notches arranged in the longitudinal axis direction, and
wherein, when one side of the virtual plane in the ridge is defined as a first side and another side of the virtual plane is defined as a second side, the specific notches having the normal vector A oriented toward a distal side on the first side and the normal vector B oriented toward a proximal side on the second side and the specific notches having the normal vector A oriented toward a distal side on the second side and the normal vector B oriented toward a proximal side on the first side are provided in the ridge in such a manner as to be alternately arranged in a longitudinal axis direction.

5. The balloon according to claim 1,
wherein the distal surface of the proximal ridge segment and the proximal surface of the distal ridge segment are closest to each other in the longitudinal axis direction at least on a line of intersection with the virtual plane.

6. The balloon according to claim 5,
wherein a separation distance in the longitudinal axis direction between the distal surface of the proximal ridge segment and the proximal surface of the distal ridge segment is formed in such a manner as to remain unchanged or to increase with increasing distance from the virtual plane.

7. The balloon according to claim 6,
wherein the separation distance in the longitudinal axis direction between the distal surface of the proximal ridge segment and the proximal surface of the distal ridge segment is formed in such a manner as to increase at least in part with increasing distance from the virtual plane.

8. The balloon according to claim 1,
wherein the distal surface of the proximal ridge segment is inclined proximally outward in the radial direction on a line of intersection with the virtual plane, and
wherein a proximal surface of the distal ridge segment is inclined distally outward in the radial direction on a line of intersection with the virtual plane.

9. The balloon according to claim 1,
wherein an angle formed between a projection vector AP of the normal vector A onto the outer surface of the balloon main body and the longitudinal axis direction toward a distal side is 20° or more and 70° or less, and
wherein an angle formed between a projection vector BP of the normal vector B onto the outer surface of the balloon main body and the longitudinal axis direction toward a proximal side is 20° or more and 70° or less.

10. The balloon according to claim 2,
wherein, in a plan view from the top portion of the ridge, a most distal portion of the distal end edge of the proximal ridge segment is on a virtual straight line passing through the top portion of the ridge and extending in the longitudinal axis direction, and/or
wherein, in a plan view from the top portion of the ridge, a most proximal portion of the proximal end edge of the distal ridge segment is on a virtual
straight line passing through the top portion of the ridge and extending in the longitudinal axis direction.

11. The balloon according to claim 2,
wherein the protruding shape of the distal end edge of the proximal ridge segment includes a curved portion curved toward a distal side, and/or
wherein the protruding shape of the proximal end edge of the distal ridge segment includes a curved portion curved toward a proximal side.

12. The balloon according to claim 2,
wherein, in a plan view from the top portion of the ridge, the protruding shape of the distal end edge of the proximal ridge segment includes a proximal first straight portion located on one side of a virtual straight line passing through the top portion of the ridge and extending in the longitudinal axis direction, and a proximal second straight portion located on another side of the virtual straight line, and
the proximal first straight portion and the proximal second straight portion extend toward a proximal side away from the virtual straight line with increasing distance, and/or
wherein, in a plan view from the top portion of the ridge, the protruding shape of the proximal end edge of the distal ridge segment includes a distal first straight portion located on one side of the virtual straight line and a distal second straight portion located on another side of the virtual straight line, and
the distal first straight portion and the distal second straight portion extend toward a distal side away from the virtual straight line with increasing distance.

13. The balloon according to claim 12,
wherein, in a plan view from the top portion of the ridge, the protruding shape of the distal end edge of the proximal ridge segment further includes a proximal third straight portion between the proximal first straight portion and the proximal second straight portion, and
the proximal first straight portion and the proximal second straight portion are located on a proximal side with respect to the proximal third straight portion, and/or
wherein, in a plan view from the top portion of the ridge, the protruding shape of the proximal end edge of the distal ridge segment further includes a distal third straight portion between the distal first straight portion and the distal second straight portion, and
the distal first straight portion and the distal second straight portion are located on a distal side with respect to the distal third straight portion.

14. The balloon according to claim 13,
wherein the proximal third straight portion and/or the distal third straight portion extends perpendicularly to the longitudinal axis direction.

15. The balloon according to claim 12,
wherein a distal end of the proximal first straight portion is connected to a distal end of the proximal second straight portion, and/or
wherein a proximal end of the distal first straight portion is connected to a proximal end of the distal second straight portion.

16. The balloon according to claim 2,
wherein, in a cross section taken along the longitudinal axis direction and passing through the top portion of the ridge, a distal outer edge of the proximal ridge segment extends in such a manner as to be inclined proximally outward in the radial direction, and/or
wherein, in a cross section taken along the longitudinal axis direction and passing through the top portion of the ridge, a proximal outer edge of the distal ridge segment extends in such a manner as to be inclined distally outward in the radial direction.

17. The balloon according to claim 1,
wherein the ridge is made of a resin, a metal, or a combination thereof.

18. A balloon catheter comprising the balloon according to claim 1.

19. The balloon according to claim 2,
wherein the ridge is made of a resin, a metal, or a combination thereof.

20. A balloon catheter comprising the balloon according to claim 2.
